(19) United States
(12) Patent Application Publication
(10) Pub. No.: US 2024/0382287 A1
(43) Pub. Date: Nov. 21, 2024

(54) SYSTEMS AND METHODS FOR GENERATING AND SCORING TEETH ALIGNER SETUPS USING COMPUTER-BASED DIGITAL DENTAL MODELS

(71) Applicant: VOYAGER DENTAL, INC., Eden Prairie, MN (US)

(72) Inventor: Michael C. MARSHALL, Eden Prairie, MN (US)

(21) Appl. No.: 18/584,693

(22) Filed: Feb. 22, 2024

Related U.S. Application Data
(60) Provisional application No. 63/486,440, filed on Feb. 22, 2023.

(51) Int. Cl. A61C 7/00; A61C 9/00; A61C 13/34; G06T 7/11; G06T 11/60; G16H 15/00

(52) U.S. Cl. CPC A61C 7/002; A61C 9/0053; A61C 13/34; G06T 7/11; G06T 11/60; G16H 15/00; G06T 2200/24; G06T 2207/30036; G06T 2210/41

(57) ABSTRACT

Disclosed are systems, methods, and techniques for determining a teeth setup score for a teeth aligner setup of a patient. The method can include: receiving, by a computer system, oral scan data for a patient including a dental impression generated by a dental impression station, image data of the patient's mouth generated by an image capture system, and/or motion data of the patient's jaw movement generated by a motion capture system, generating a teeth aligner setup for the patient based on the oral scan data, simulating movement of one or more teeth of the patient using the teeth aligner setup, generating a movement report based on the simulated movement, determining a teeth setup score for the teeth aligner setup based on providing the movement report as input to a machine learning model, and returning the teeth setup score for presentation in a computing device's GUI display for a relevant user.

### CROSS-REFERENCE TO RELATED APPLICATIONS

This application claims the priority benefit of U.S. Provisional Patent Application No. 63/486,440, filed Feb. 22, 2023, which is incorporated herein by reference in its entirety.

### TECHNICAL FIELD

This document generally describes devices, systems, and methods related to computer-based modeling and scoring of a tooth aligner setup for a patient using a digital dental model of the patient.

### BACKGROUND

A teeth aligner is a plastic replica of a patient's teeth. Teeth aligners can be worn to help straighten the patient's teeth and/or maintain the patient's teeth in a desired position and/or setup. The teeth aligners can be worn while the patient sleeps, over an extended period of time, and/or for some prescribed amount of time. By wearing the teeth aligners, gentle pressure can be applied on the patient's teeth to reposition and then maintain the teeth at a desired teeth setup that is achievable with the design and fit of the teeth aligners. In other words, the teeth aligners can be designed to apply desired forces on one or more teeth to move those teeth to a desired teeth setup. The teeth aligners can be set up and prescribed by an orthodontist or other care provider. Teeth aligners can be formed according to a position of one or more teeth in the patient's mouth in a next staged location. The teeth can continue to move until the teeth line up with the aligner.

The teeth aligners can be designed by the prescribing orthodontist or other care provider. The orthodontist can, for example, take an imprint of the patient's teeth and manually determine how much one or more of the patient's teeth, according to the imprint, can be moved to achieve a desired teeth setup (e.g., straight teeth). The orthodontist can then design an aligner setup based on the amount of movement needed to move the patient's teeth to the desired setup. Once the teeth aligner setup is established, the setup can be used to manufacture and fabricate the teeth aligners for the patient. Sometimes, the teeth aligner setup can be sent to one or more different aligner-manufacturing companies, all of whom may manufacture the teeth aligners according to the setup with varying degrees of similarity and/or accuracy. In other words, the aligner-manufacturing companies and even the prescribing orthodontist or other healthcare provider may not follow a standardized process for designing, manufacturing, and fabricating teeth aligners.

### SUMMARY

The document generally describes technology for designing and scoring teeth aligner setups using a digital dental model for a patient. More particularly, a digital dental model can be generated for a patient based on at least oral scan data. Using the digital dental model, a practitioner, such as an orthodontist, can determine a preferred or target teeth setup for the patient. The disclosed technology can implement various rules, algorithms, and/or machine learning models to determine 6 degrees of movement for the patient's teeth to move from a current setup to the preferred or target setup. The movement can be achieved with a teeth aligner, which can be designed according to the disclosed techniques. Data such as the digital dental model, the preferred or target teeth setup, and/or the movement data can be provided as input to a machine learning model that was trained to correlate such data and generate a single metric or score that quantifies a difficulty level for moving the teeth to the preferred or target setup with teeth aligners. This metric or score can be used by the practitioner to determine whether the patient is a good candidate for receiving a teeth aligner setup and/or whether a teeth aligner setup would help achieve a desired or best teeth setup for the particular patient. The metric or score can also include various annotations or other information indicating rationale behind the metric or score value, such as whether the teeth aligner setup would cause a threshold amount of movement of the patient's molars that is unsafe or otherwise not preferred.

The determined metric or score can be used by the practitioner, other relevant stakeholders, and/or computing systems to generate a preferred teeth aligner setup for the particular patient. The teeth aligner setup can then be accurately manufactured/fabricated for the patient. Typically, a teeth aligner setup can be determined by a practitioner, such as an orthodontist, upon assessing a patient's teeth. The teeth aligner setup can be sent to an entity that manufactures an aligner for the patient based on the setup. Sometimes, the entity can also determine the teeth aligner setup and then manufacture an aligner based on the setup. The entity, practitioner, and other relevant stakeholders, such as other aligner-determining and/or manufacturing entities, may not follow a standardized process for designing and manufacturing aligners. Therefore, every aligner can be assessed and designed differently, resulting in vastly different aligners being made for a particular patient. Because of the lack of a standardized process, it can be challenging to determine a best teeth aligner setup for the particular patient.

One or more embodiments described herein include a method for determining a teeth setup score for a teeth aligner setup of a patient, the method including: receiving, by a computer system, oral scan data for a patient, the oral scan data including at least one of (i) a dental impression generated by a dental impression station, (ii) image data of the patient's mouth generated by an image capture system, and (iii) motion data of the patient's jaw movement generated by a motion capture system, generating, by the computer system, a teeth aligner setup for the patient based on the oral scan data, simulating, by the computer system, movement of one or more teeth of the patient using the teeth aligner setup, generating, by the computer system, a movement report based on the simulated movement, determining, by the computer system, a teeth setup score for the teeth aligner setup based on providing the movement report as input to a machine learning model, and returning, by the computer system, the teeth setup score for presentation in a graphical user interface (GUI) display of a computing device of a relevant user.

The embodiments described herein can optionally include one or more of the following features. For example, returning, by the computer system, the teeth setup score further can include generating instructions that, when executed by the computing device, cause the computing device to: output, in the GUI display, the teeth setup score and the teeth aligner setup, the teeth aligner setup being displayed as visually overlaying a digital dental model of the patient's mouth, receive user input indicating at least one modification to the teeth aligner setup, and transmit the user input to the computer system. The method can also include receiving, by the computer system and from the computing device, the user input, adjusting, by the computer system, the teeth aligner setup based on the user input, re-simulating, by the computer system, movement of the patient's teeth using the adjusted teeth aligner setup, and determining, by the computer system, an updated teeth setup score for the adjusted teeth aligner setup. Outputting the teeth setup score can also include outputting information about at least one tooth according to the movement report that had a greatest impact on the determined teeth setup score.

As another example, the method can further include generating, by the computer system, a digital dental model for the patient based on the oral scan data. The teeth setup score can indicate a difficulty level that corresponds to performing an alignment procedure on the patient with the teeth aligner setup. A higher teeth setup score can indicate a greater difficulty level with performing an alignment procedure on the patient with the teeth aligner setup and a lower teeth setup score can indicate a lower difficulty level with performing the alignment procedure on the patient with the teeth aligner setup. The method can also include generating, by the computer system, instructions that, when executed by a rapid fabrication machine, causes the rapid fabrication machine to manufacture a teeth aligner dental appliance for the patient based on the teeth aligner setup. The relevant user can be at least one of a healthcare provider, dentist, orthodontist, and nurse.

As another example, simulating, by the computer system, movement of one or more teeth of the patient using the teeth aligner setup can include simulating movement of the patient's teeth from a pre-treatment state to a post-treatment state, the pre-treatment state being a current state of the patient's teeth according to the oral scan data and the post-treatment state being achieved using the teeth aligner setup. The movement report can include at least one of: mesial-distal translation data, buccal-lingual translation data, occlusal-gingival translation data, rotation change data, tip change data, and torque change data. The movement report can include data about at least one degree of freedom of movement for each tooth in the oral scan data for the patient. Sometimes, the method can also include applying, by the computer system, a coordinate system to each tooth in the oral scan data for the patient, the at least one degree of freedom of movement for each tooth being determined relative the coordinate system applied to the tooth.

As yet another example, the method can also include determining, by the computer system, whether the teeth setup score exceeds a threshold score value, generating, by the computer system and based on a determination that the teeth setup score exceeds the threshold score value, at least one recommendation for adjusting the teeth aligner setup, where adjusting the teeth aligner setup can cause at least one change in teeth movement that results in a lower teeth setup score than the teeth setup score that exceeds the threshold score value, and returning, by the computer system, the at least one recommendation for presentation in the GUI display of the computing device. The method can also include automatically adjusting, by the computer system and responsive to determining that the teeth setup score exceeds a second threshold score value, the teeth aligner setup based on the at least one recommendation, the second threshold score value being higher than the threshold score value.

In some implementations, generating, by the computer system, a teeth aligner setup for the patient based on the oral scan data can include: segmenting each tooth in the oral scan data, identifying critical landmarks for each segmented tooth, generating the teeth aligner setup based on the critical landmarks, defining a wire plane through the segmented teeth in the teeth aligner setup, applying a coordinate system to each segmented tooth in the teeth aligner setup, and aligning each coordinate system with a base of each segmented tooth, the base of the segmented tooth being defined by the wire plane. In some implementations, the machine learning model was trained, by the computer system, using a process that can include: receiving training data of teeth aligner setups for a plurality of patients, annotating the training data with at least one of (i) difficulty scores having relationships with the teeth aligner setups and (ii) attributes that correspond to the difficulty scores, training the model to score a teeth aligner setup based on the annotated training data, and returning the model for runtime execution.

One or more embodiments described herein can include a system for scoring a teeth aligner setup for a patient, the system including: a computing device having processors and memory, where the computing device can be configured to: present, in a graphical user interface (GUI) display, a digital dental model of a patient, the digital dental model representing a post-treatment teeth setup for the patient, present, in the GUI display, a teeth aligner setup as a graphical visual overlaying at least a portion of the digital dental model, the teeth aligner setup representing a pre-treatment teeth setup for the patient, present, in the GUI display and for each tooth in the teeth aligner setup, a coordinate system, transmit, to a computing system, a request for a teeth setup score based on the teeth aligner setup overlaying the digital dental model, receive, from the computing system, data indicating at least the teeth setup score, the teeth setup score being determined, by the computing system, based on (i) simulating movement of teeth in the teeth aligner setup to teeth in the digital dental model representing the post-treatment teeth setup for the patient to generate a teeth movement report and (ii) providing the teeth movement report as input to a machine learning model that was trained to generate the teeth setup score, and present, in the GUI display, the teeth aligner setup as visually overlaying a different portion of the digital dental model, the different portion of the digital dental model being determined based on the teeth movement report, and present, in the GUI display the teeth setup score.

The system can optionally include one or more of the abovementioned features and/or one or more of the following features. For example, the teeth in the teeth aligner setup can be aligned with the teeth in the digital dental model when the teeth aligner setup visually overlays the different portion of the digital dental model. As another example, the computing device can receive second user input indicating at least one adjustment to the teeth aligner setup based on the teeth setup score, transmit the second user input to the computing system, receive, from the computing system, data indicating (i) an adjusted teeth aligner setup based on the user input and (ii) an updated teeth setup score based on re-simulating movement of the teeth in the adjusted teeth aligner setup relative the teeth in the digital dental model, and present, in the GUI display, the adjusted teeth aligner setup as visually overlaying the digital dental model and the updated teeth setup score. The teeth setup score can be presented in at least one of (i) another GUI display and (ii) a pop-out window overlaying at least a portion of the GUI display. The computing device can also be configured to present, in the GUI display, at least one of: (i) a recommendation of whether the teeth aligner setup should be used for the patient, the recommendation being based on whether the teeth setup score satisfies threshold alignment criteria, (ii) a difficulty level associated with performing an alignment procedure with the teeth aligner setup, the difficulty level being a string value that corresponds to a numeric value of the teeth setup score, and (iii) a score explanation indicating at least one movement associated with at least one tooth of the teeth aligner setup that satisfied threshold movement criteria.

The disclosed technology can provide one or more of the following advantages. For example, the disclosed technology provides for more efficiently processing various information and data about the patient's teeth and movement of their teeth to determine and score an accurate teeth aligner setup for the particular patient. The disclosed techniques can distill a set of complex information to a single metric, such as a single score or multiple scores (e.g., complexity of movement, impact on root health, etc.), that can be used to assessing a teeth aligner setup. The disclosed technology similarly provides improved and effective machine learning techniques for accurately determining 6 degrees of movement of the patient's teeth to score a teeth aligner setup for the patient.

Additionally, the disclosed technology provides a standardized process with an established movement scale that can be consistently applied to generate and score teeth aligner setups across different entities. The disclosed technology standardizes an inherently subjective process for determining movement of teeth with certain aligner setups. The disclosed technology also helps practitioners in dentistry and orthodontics better and more accurately perform assessments about teeth movement and whether a patient is a good candidate for an aligner setup.

The details of one or more implementations are set forth in the accompanying drawings and the description below. Other features and advantages will be apparent from the description and drawings, and from the claims.

### BRIEF DESCRIPTION OF THE DRAWINGS

FIG. 1A is conceptual diagram of a system for scoring a teeth setup using a digital denture model.

FIG. 1B is a schematic block diagram of the system of FIG. 1A for fabricating a motion-based aligner using a digital dental model.

FIG. 2 is a schematic block diagram illustrating an example motion capture system for capturing jaw movement.

FIG. 3 illustrates a block diagram of an example patient assembly of FIG. 2.

FIG. 4 illustrates an example implementation of the clutch of FIG. 3.

FIGS. 5A-B are cross-sectional side views that illustrate attachment of a dentition coupling device of the clutch or reference structure of FIG. 2 to a dental implant.

FIG. 6 is an example of the motion capture system of FIGS. 1A-B in which two screens are used.

FIG. 7 illustrates a top view of a reference structure of FIG. 3 and the imaging system of FIGS. 1A-B.

FIG. 8 illustrates a perspective view of the reference structure of FIG. 7 disposed between the screens of the imaging system of FIG. 7.

FIG. 9 is a flowchart of an example process for fabricating a teeth aligner setup for a patient.

FIGS. 10A-B is a flowchart of a process for generating and scoring a teeth aligner setup for a patient.

FIG. 11 is a flowchart of a process for training a model for scoring a teeth aligner setup of a patient.

FIG. 12A illustrates an example graphical user interface (GUI) display for viewing a digital dental model of a patient's top teeth with an overlaid teeth aligner setup.

FIG. 12B illustrates an example GUI display for viewing a treatment outcome and teeth aligner setup score for the patient's top teeth shown in FIG. 12A.

FIG. 12C illustrates an example GUI display for viewing a digital dental model of a patient's bottom teeth with an overlaid teeth aligner setup.

FIG. 12D illustrates an example GUI display for viewing a treatment outcome and teeth aligner setup score for the patient's bottom teeth shown in FIG. 12C.

FIG. 12E illustrates an example GUI display for receiving user input to adjust a teeth aligner setup to achieve a desired treatment outcome.

FIG. 13 is a conceptual diagram illustrating runtime execution of a teeth aligner setup scoring model using the disclosed techniques.

FIG. 14 is a schematic diagram that shows an example of a computing device and a mobile computing device.

Like reference symbols in the various drawings indicate like elements.

### DETAILED DESCRIPTION OF ILLUSTRATIVE IMPLEMENTATIONS

This document generally relates to technology for designing and scoring teeth aligner setups using a digital dental model for a patient. More particularly, a digital dental model can be generated for a patient based on at least oral scan data and motion data. Using the digital dental model, a practitioner, such as an orthodontist, can determine a preferred or target teeth setup for the patient that can be achieved with a teeth aligner. The disclosed technology can implement various rules, algorithms, and/or machine learning models to determine how much movement the patient's teeth would make in order to achieve the preferred setup with the teeth aligner. Movement data can then be provided as input to a machine learning model to score a setup for the teeth aligner. The score can be used by the practitioner to determine whether the patient is a good candidate for receiving the teeth aligner and/or whether achieving the preferred teeth setup would be challenging, difficult, or otherwise not recommended with the teeth aligner setup. The score can also be used to determine, by the practitioner or automatically by a computing system, one or more adjustments to the teeth aligner setup that can improve the score. The disclosed technology can also include various graphical user interface (GUI) displays presentable at computing devices of relevant users, such as practitioners or orthodontists, that can be used to visualize the digital dental model, the preferred teeth setup for the patient, the teeth aligner setup, an impact of the teeth aligner setup on moving the patient's teeth in the digital dental model to the preferred teeth setup, and scoring information for the teeth aligner setup. The GUIs can also present selectable and interactive features for the relevant user to design and/or adjust the teeth aligner setup and/or the preferred teeth setup for the particular patient. The GUIs can also present selectable and interactive features for the relevant user to identify types of movement that are acceptable, exclude one or more other types of movement, identify teeth that can be moved, identify teeth that should not be moved, and otherwise provide input that causes a computing system to automatically adjust the teeth aligner setup and score the adjusted teeth aligner setup. As a result, the disclosed technology can be used to improve and standardize a process of designing, assessing, and fabricating teeth aligners for particular patients.

As described herein, a motion-based digital denture design system may be used to capture actual motion data from the patient to aid in the design of aligners and other dental appliances, such as dentures, dental prosthetics, crowns, etc. The motion data may provide for aligners that fit the patient better than aligners that are designed using different standards based on the designing and/or manufacturing entity.

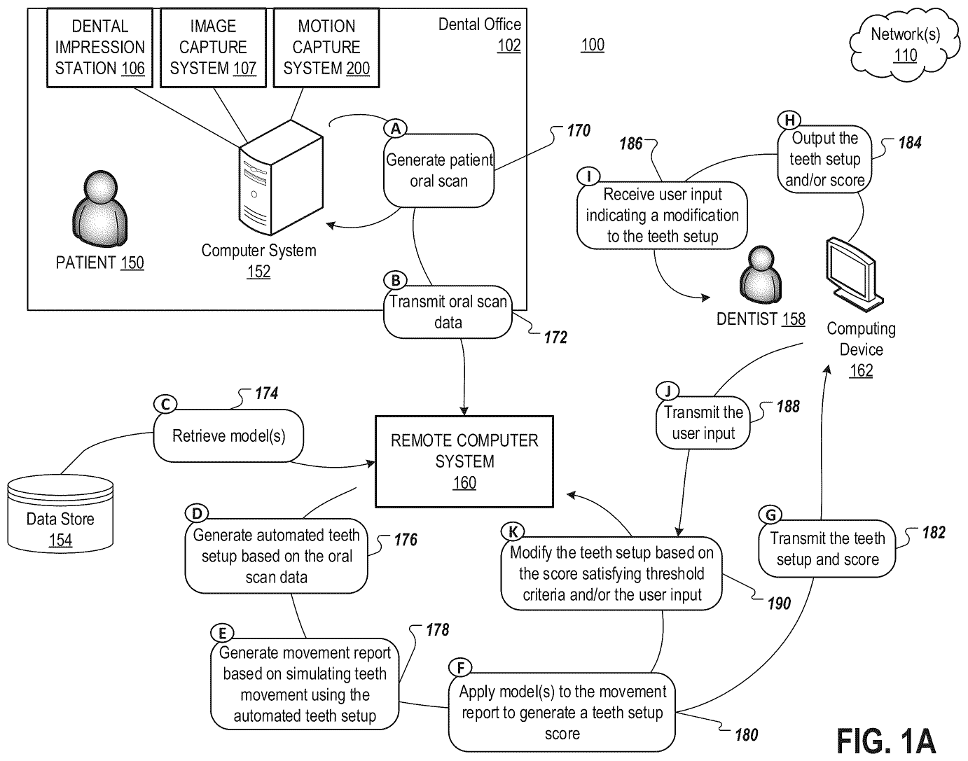

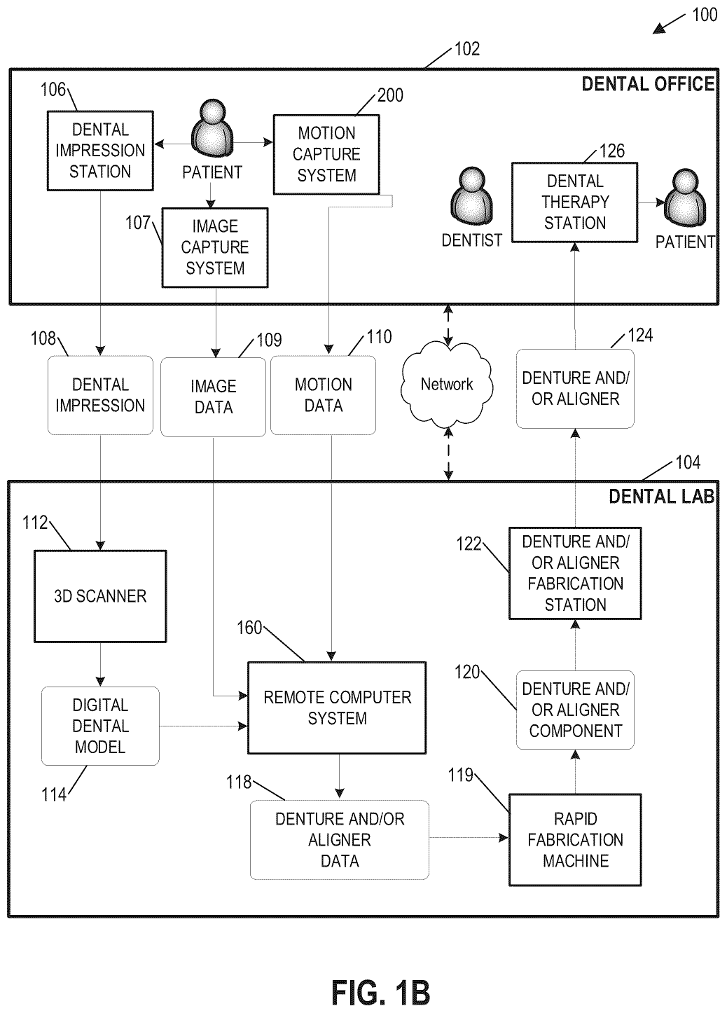

Referring to the figures, FIG. 1A is conceptual diagram of a system 100 for scoring a teeth setup using a digital denture model. In the system 100, a computer system 152 may communicate (e.g., wired and/or wireless) with a remote computer system 160 and data store 154 via network(s) 110. The computer system 152 can be any type of computing device, network of computing devices and/or systems, and/or cloud-based system described herein. The computer system 152 can be configured to collect and/or capture data about a patient 150 in a dental office 102. The remote computer system 160 can be any type of computing device, network of computing devices and/or systems, and/or cloud-based system described herein. The remote computer system 160 can be configured to receive data from the computer system 152 and use the data to generate and assess teeth aligner setups for the patient 150. Sometimes, the computer system 152 and the remote computer system 160 can be a same computer system.

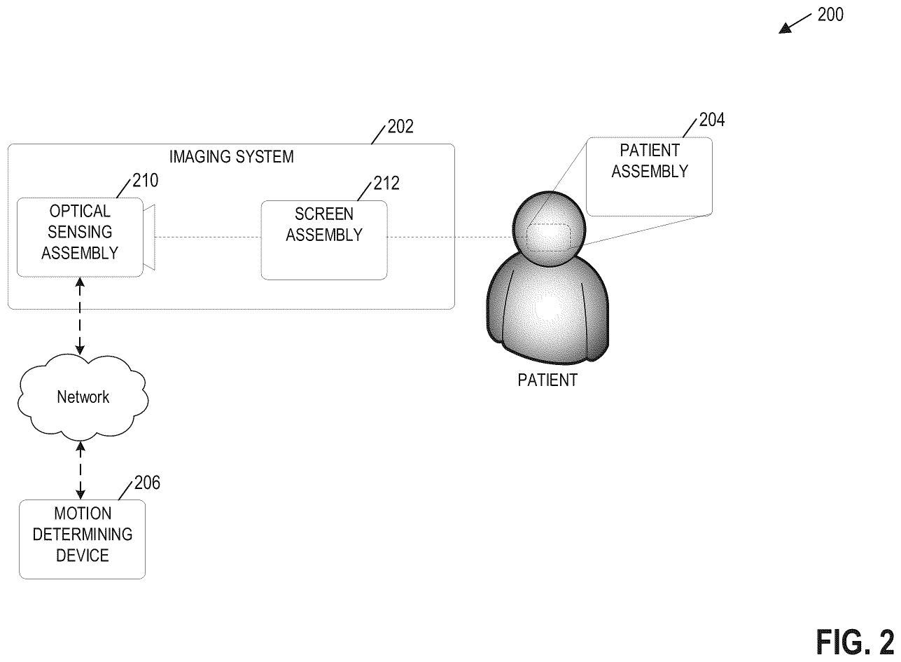

The computer system 152 can communicate via the network(s) 110 with a dental impression station 106, an image capture system 107, and a motion capture system 200. Sometimes, the computer system 152 can be integrated into or otherwise part of at least one of the dental impression station 106, the image capture system 107, and the motion capture system 200. The computer system 152 can generate instructions that cause the dental impression station 106, the image capture system 107, and/or the motion capture system 200 to capture data of the patient 150. Refer to FIGS. 1B-2 for further discussion about the dental impression station 106, the image capture system 107, and the motion capture system 200.

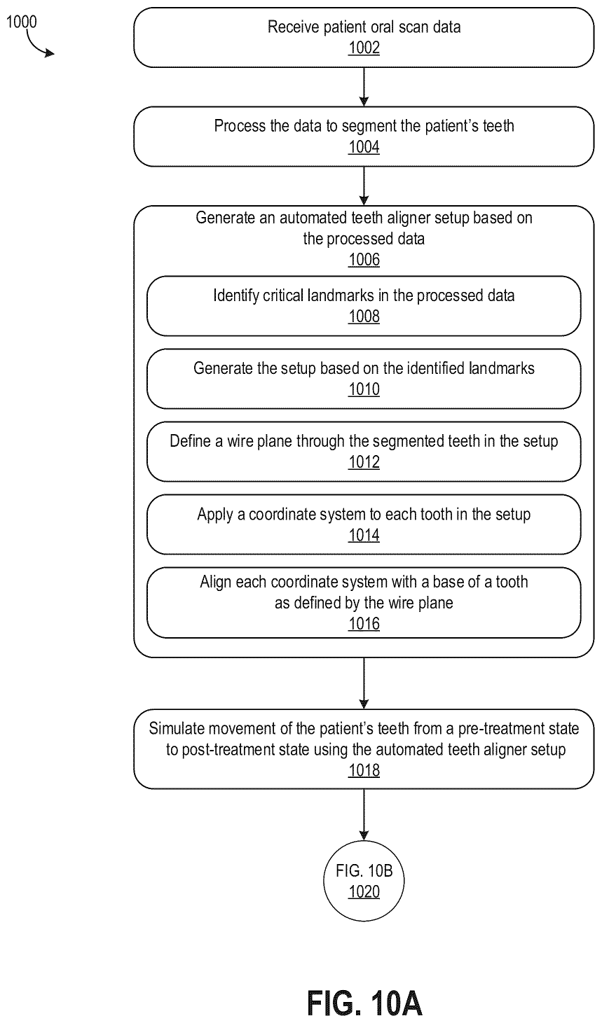

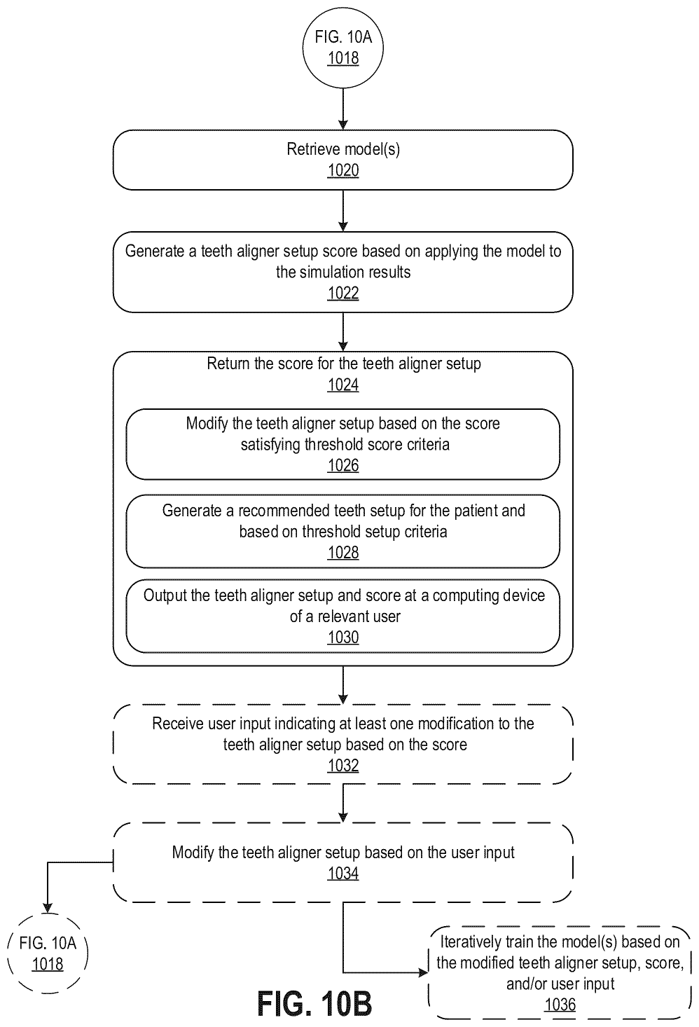

The remote computer system 160, as mentioned above, can be configured to automatically design digital dental models for patients, such as the patient 150, based on data collected by the computer system 152. The system 160 can also design/generate teeth aligner setups and assess the teeth aligner setups to determine whether such setups achieve desired teeth setup outcomes for patients. Refer to FIGS. 10-13 for further discussion about techniques performed by the remote computer system 160.

The data store 154 can be any type of database, data store, cloud-based storage, and/or data repository that is configured to store information about digital dental models, patients, teeth aligner setups, and/or teeth aligner setup assessments. The data store 154 can store the abovementioned information in association with particular patients, such as a digital dental model for the patient 150, a preferred or target teeth setup for the patient 150, a teeth aligner setup for the patient 150, and a computer-generated assessment of the teeth aligner setup for the patient 150. The data store 154 can also store information that is generic and applicable to variety of patients.

Still referring to FIG. 1A, the computer system 152 can generate a patient oral scan for the patient 150 in block A (170). For example, the computer system 152 can capture and/or receive patient dental data. The data can include dental impression data, images of the patient 150's mouth, and/or motion data, all of which may be captured and generated by the respective dental impression station 106, the image capture system 107, and the motion capture system 200. As described herein, the captured data can include oral scan data of the patient 150's mouth. Refer to FIG. 1B for further discussion about capturing the patient dental and/or denture data.

In block B, the computer system 152 can transmit the oral scan data to the remote computer system 160 (172). Sometimes, the oral scan data can be stored in the data store 154 and then retrieved by the remote computer system 160 at a later time for further processing.

The remote computer system 160 can also retrieve one or more models in block C (174).

In some implementations, block C (174) can be performed in one or more other orders. For example, block C can be performed before block F (180), which is described further below. One or more other orders of operations are also possible.

In block D (176), the remote computer system 160 can generate an automated teeth setup based on the oral scan data. The remote computer system 160 can generate a digital dental model for the patient based on the oral scan data, as part of block D. Refer to FIGS. 1B-9 for further discussion about generating the digital dental model. The remote computer system 160 can also generate a teeth aligner setup, which can be applied to the digital dental model for the patient 150 to determine movement of the patient's teeth. Refer to FIGS. 10A-B for further discussion about generating the teeth aligner setup. In some implementations, the remote computer system 160 can also generate or otherwise determine a preferred or target teeth setup, which can be achieved by applying the teeth aligner setup to the digital dental model for the patient 150.

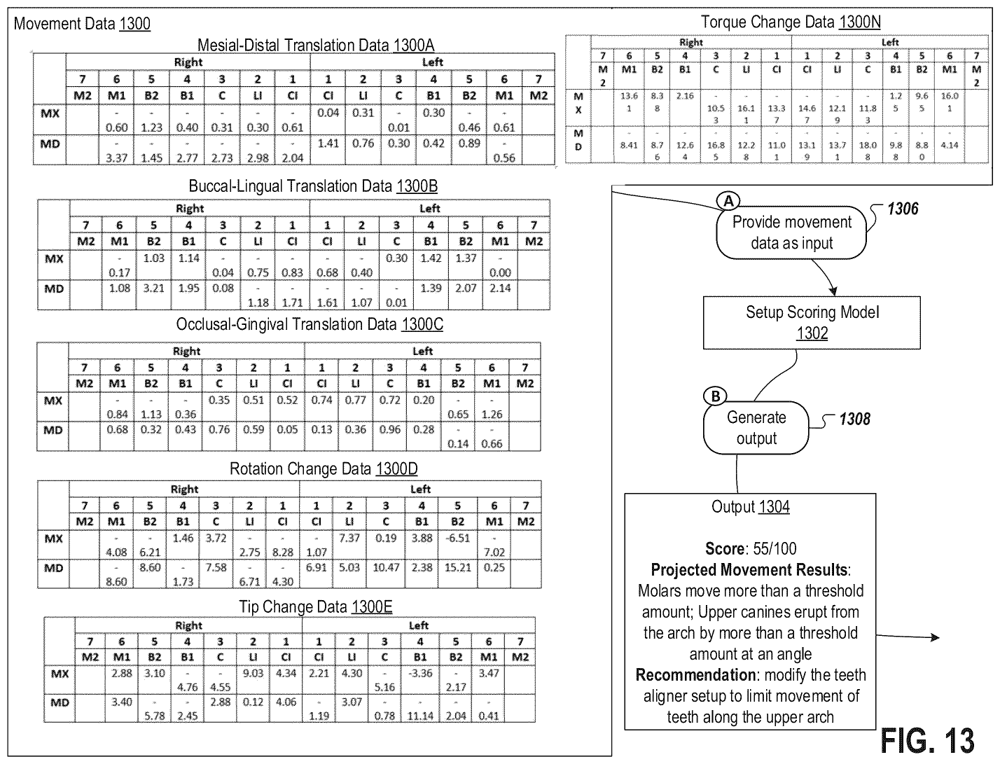

The remote computer system 160 generates a movement data report in block E (178) based on simulating teeth movement using the automated teeth setup. Block E can be performed using one or more machine learning techniques, algorithms, and/or models. For example, the remote computer system 160 can provide the digital dental model, the teeth aligner setup, and/or the target teeth setup for the patient 150 as input to a machine learning model. The machine learning model can be trained to generate output indicating how much each tooth (or one or more predefined or particular teeth) moves based on applying the teeth aligner setup to the digital dental model for the patient 150. Refer to FIG. 13 for further discussion about generating the movement report.

The remote computer system 160 applies at least one model to the movement report to generate a teeth setup score (block F, 180). For example, the system 160 can provide the movement report and optionally other information (e.g., the digital dental model for the patient 150, the teeth aligner setup, the target teeth setup, and/or other patient-specific data/information, such as which teeth can or should not be moved) as input to the model. The model can be trained to generate a singular metric, such as the score, as output based on correlating, aggregating, and/or assessing the combination of model inputs. The teeth setup score can be a numeric value, string value, Boolean value, or other predefined value. A standardized scale or other scoring criteria can be used to ensure that all automated teeth setups are similarly assessed and scored. As an illustrative example, the teeth setup score can be a numeric value on a scale ranging from 0 to 100. The teeth setup score can indicate a difficulty level for applying the teeth aligner setup to the patient 150's teeth in order to achieve the target teeth setup for the patient 150. In block F (180), the remote computer system 160 may also generate one or more annotations or other information indicating various features that contributed to the teeth setup score value. For example, a higher teeth setup score can indicate a higher difficulty level (and therefore a less desirable teeth aligner setup to achieve the target teeth setup for the patient 150). The higher teeth setup score can be attributed to the teeth aligner setup causing one or more molars of the patient 150 to move beyond a threshold amount of movement. The threshold amount of movement can indicate, for the particular patient 150 or a general population of patients, how much adjustment to position, angle, torque, rotation, etc. of the molars is acceptable before the adjusted molars negatively impact the health, wellbeing, safety, and/or positioning of other teeth in the patient 150's mouth. Refer to FIGS. 10-13 for further discussion about scoring the teeth setup.

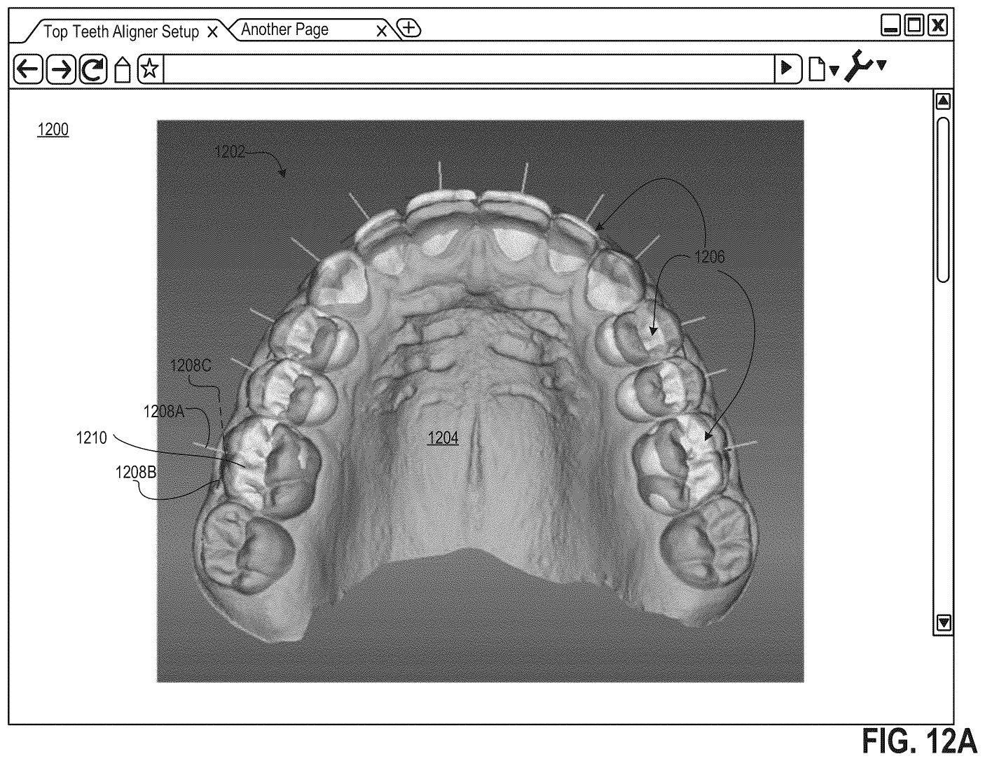

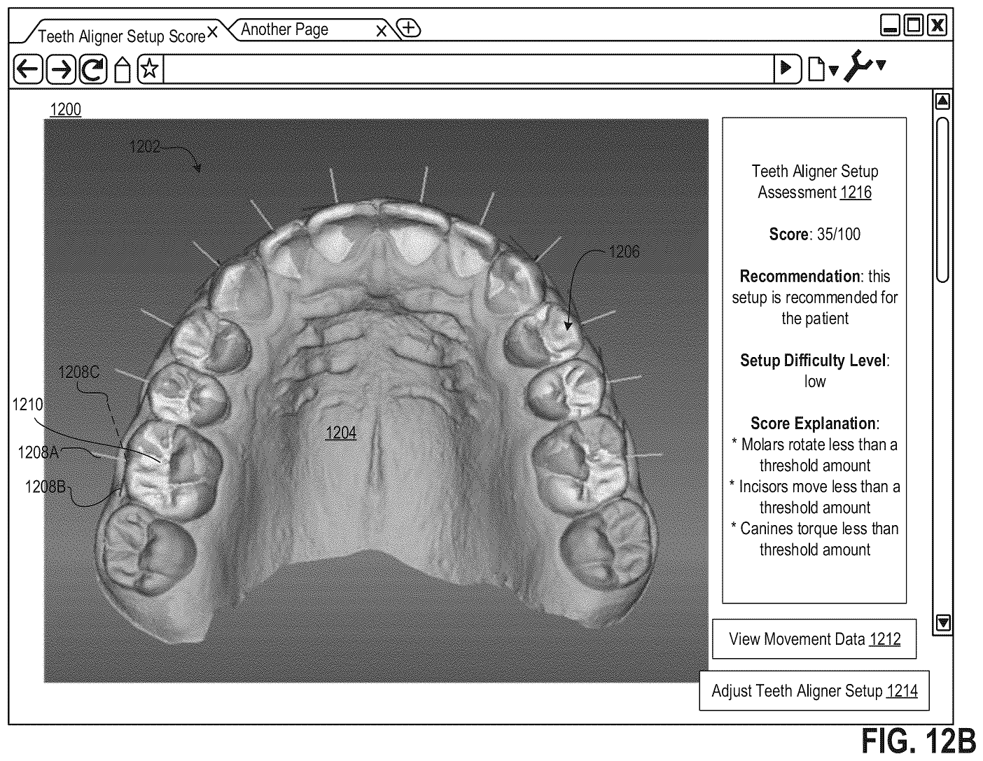

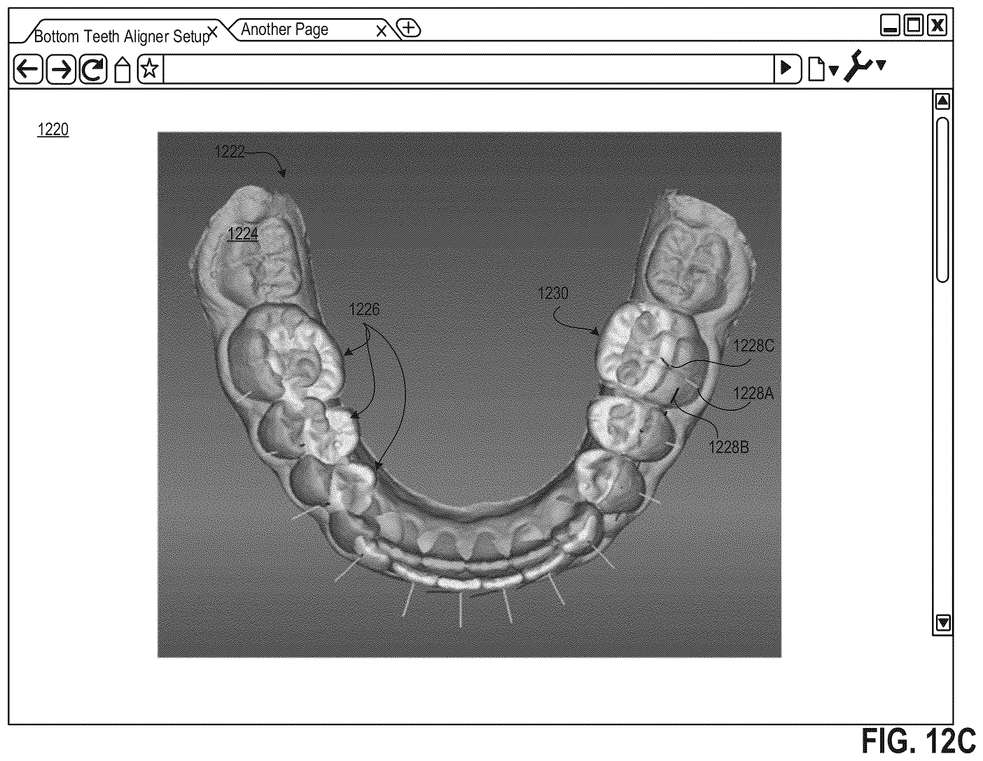

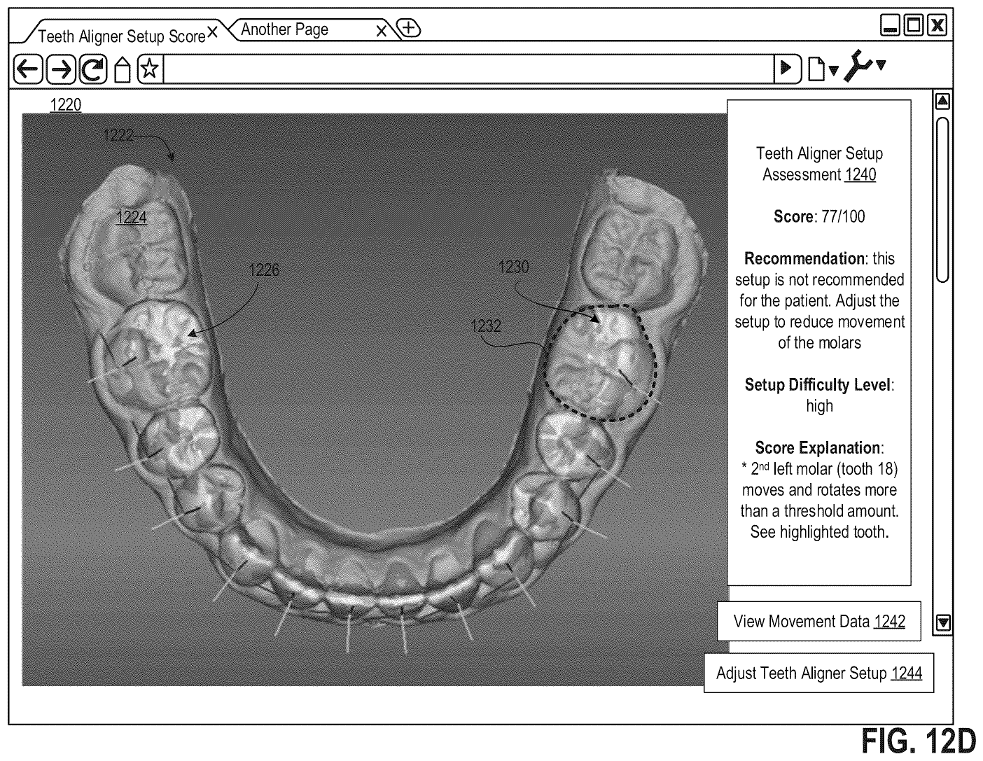

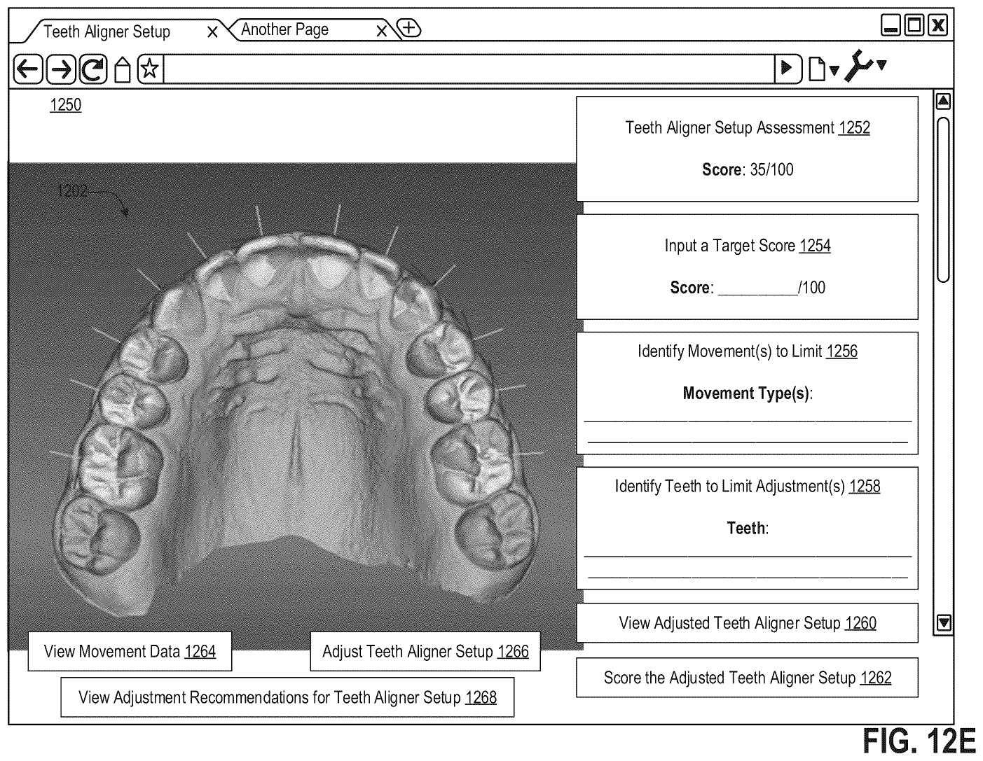

The remote computer system 160 can transmit at least the teeth setup and score to a computing device 162 of a dentist 158, or other relevant user/care provider (block G, 182). The computing device 162 can be any type of computing system, including but not limited to a mobile device, mobile phone, smartphone, cellphone, laptop, tablet, and/or computer. The computing device 162 can output the teeth setup and/or the score in a GUI display for presentation to the dentist 158 (block H, 184). The dentist 158 can review the teeth setup and/or the score to determine whether to modify the teeth aligner setup and/or the target teeth setup. The dentist 158 can also use the outputted information to determine whether to apply the teeth aligner setup to the particular patient 150 and/or whether to have the teeth aligner setup sent to a rapid fabrication machine (e.g., refer to FIG. 1B) to fabricate and manufacture a teeth aligner for the patient 150 according to the teeth aligner setup. Accordingly, the computing device 162 can receive user input indicating at least one modification to the teeth setup in block I (186). The user input can then be transmitted by the computing device 162 to the remote computer system 160 (block J, 188). Refer to FIGS. 12A-E for further discussion about information that is outputted at the computing device 162.

Once the remote computer system 160 receives the user input, the system 160 can modify the teeth setup (block K, 190). The system 160 can modify the teeth setup based on the teeth setup score satisfying some threshold criteria. As an illustrative example, if the score is greater than a threshold value, such as 75/100, which indicates a high difficulty level for applying the teeth aligner setup to achieve the target teeth setup for the patient 150, the system 160 can automatically generate one or more recommendations for changing the teeth aligner setup and/or the target teeth setup to bring the score lower than another threshold value (such as 65/100, which can indicate that the teeth aligner setup may provide a best outcome for the particular patient 150). The recommendations can include causing one or more predetermined adjustments in one or more degrees of freedom for one or more teeth in the digital dental model for the patient 150. The recommendations can be presented to the dentist 158 at the computing device 162 for review and approval. In some implementations, the recommendations can be automatically implemented by the remote computer system 160. When the recommendations are implemented, the remote computer system 160 can repeat one or more of the blocks D-K (176-190). One or more of the blocks D-K (176-190) can be repeated until a desired teeth setup score is achieved and/or the dentist 158 determines that the teeth aligner setup and/or the target teeth setup are the best setups that can be achieved for the patient 150.

FIG. 1B is a schematic block diagram of the system 100 of FIG. 1A for fabricating a motion-based aligner using a digital dental model. In this example, the system 100 includes the dental office 102 of FIG. 1A and a dental lab 104. The example dental office 102 includes the motion capture system 200 (described further with respect to at least FIG. 2), the dental impression station 106, the image capture system 107, and a dental therapy station 126. Although shown as separate components, the image capture system 107 may be a sub-component of the motion capture system 200 (as described elsewhere). Although shown as a single dental office 102, in some implementations, the dental office 102 includes multiple dental offices. For example, one or more of the dental impression station 106, the image capture system 107, and the motion capture system 200 can be in a different dental office than the dental therapy station 126. Further, one or more of the dental impression station 106, the motion capture system 200, and the dental therapy station 126 may not be located in a dental office.

The example dental impression station 106 is configured to generate a dental impression 108 of dentition of a patient (e.g., the patient 150 in FIG. 1A). The dental impression 108 is a geometric representation of the dentition of the patient, which may include teeth (if any) and edentulous (gum) tissue, or gingiva as described herein. In some implementations, the dental impression 108 is a physical impression captured using an impression material, such as sodium alginate, polyvinylsiloxane or another impression material.

In some implementations, the dental impression 108 is a digital impression. The digital impression may be represented by one or more of a point cloud, a polygonal mesh, a parametric model, or voxel data. The digital impression can be generated directly from the dentition of the patient, using for example an intraoral scanner. Example intraoral scanners include the TRIOS Intra Oral Digital Scanner, the Lava Chairside Oral Scanner C.O.S., the Cadent iTero, the Cerec AC, the Cyrtina IntraOral Scanner, and the Lythos Digital Impression System from Ormco. In other implementations, a digital impression is captured using other imaging technologies, such as computed tomography (CT), including cone beam computed tomography (CBCT), ultrasound, and magnetic resonance imaging (MRI). In yet other implementations, the digital impression is generated from a physical impression by scanning the impression or plaster model of the dentition of the patient created from the physical impression. Examples of technologies for scanning a physical impression or model include three-dimensional laser scanners and computed tomography (CT) scanners. In yet other implementations, digital impressions can be created using other technologies.

The motion capture system 200 is configured to capture a representation of movement of dental arches relative to each other in the patient's mouth. In some implementations, the motion capture station generates motion data 110. The dental impression 108 can also be used to generate a patient-specific dentition coupling device for capturing patient motion using the motion capture system 200. Some implementations described herein may use other types of motion capture systems to generate motion data of the patient's mouth.

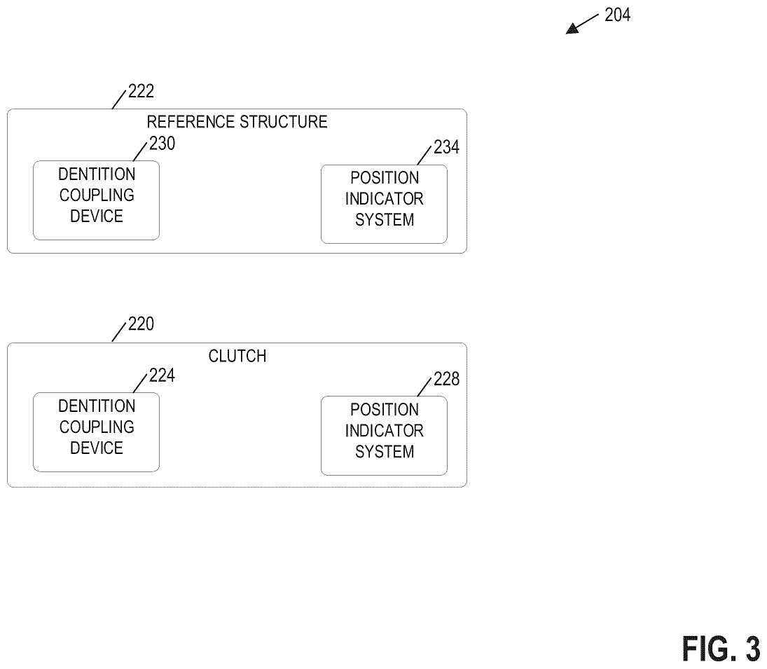

In some implementations, the motion capture system 200 generates the motion data 110 from optical measurements of the dental arches that are captured while the dentition of the patient is moved. The optical measurements can be extracted from image or video data recorded while the dentition of the patient is moved. Additionally, the optical measurements can be captured indirectly. For example, the optical measurements can be extracted from images or video data of one or more devices (e.g., a patient assembly such as the patient assembly 204 that is illustrated and described with respect to at least FIGS. 2-3) that are secured to a portion of the dentition of the patient. The motion data 110 can be generated using other processes as well. Further, the motion data 110 may include transformation matrices that represent position and orientation of the dental arches. The motion data 110 may include a series of transformation matrices that represent various motions or functional paths of movement for the patient's dentition. Other implementations of the motion data 110 are possible as well.

Still images can be captured of the patient's dentition while the dentition of the patient is positioned in a plurality of bite locations. Image processing techniques can then be used by any of the disclosed computing systems to determine positions of the patient's upper and lower arches relative to each other (either directly or based on the positions of the attached patient assembly 204). In some implementations, the motion data 110 can be generated by interpolating between the positions of the upper and lower arches determined from at least some of the captured images.

The motion data 110 may be captured with the patient's jaw in various static positions or moving through various motions. For example, the motion data 110 may include a static measurement representing a centric occlusion (e.g., the patient's mandible closed with teeth fully engaged) or centric relation (e.g., the patient's mandible nearly closed, just before any shift occurs that is induced by tooth engagement or contact) bite of a patient. The motion data 110 may also include static measurements or sequences of data corresponding to protrusive (e.g., the patient's mandible being shifted forward while closed), lateral excursive (e.g., the patient's mandible shifted/rotated left and right while closed), hinging (e.g., the patient's mandible opening and closing without lateral movement), chewing (e.g., the patient's mandible chewing naturally to, for example, determine the most commonly used tooth contact points), and border movements (e.g., the patient's mandible is shifted in all directions while closed, for example, to determine the full range of motion) of the patient's jaw. In some implementations, the motion data is captured while the patient is using a Lucia jig or leaf gauge so that the patient's teeth (for patients who are not completely edentulous) do not impact/contribute to the movement data. This motion data 110 may be used to determine properties of the patient's temporomandibular joint (TMJ). For example, hinging motion of the motion data 110 may be used to determine the location of the hinge axis of the patient's TMJ.

In some implementations, a representation of the motion of the hinge axis may be displayed while the motion data 110 is being captured. For example, a computing device may cause a line segment to be displayed in relation to a representation of the patient's dentition. The line segment may be displayed at a location that is approximately where the patient's condyle is located. The line segment may move in concert with the relative motion of the patient's mandible (lower dentition). Visually, the movement of the line may appear to rotate at a location approximately equal to the hinge axis of the patient's TMJ. Furthermore, during motion capture the caregiver may annotate the motion data to identify portions of the motion data such as the motion data corresponding to hinging open/closed. For example, the caregiver may actuate an input such as a button on a user interface, a physical button, or a foot pedal to annotate portions of the motion data.

The image capture system 107 is configured to capture image data 109 of the patient. The image data 109 may include one or more static images or videos of the patient. The static images or frames with the image data 109 may be associated with the motion data 110. For example, a specific image from the image data 109 may be associated with a specific frame of the motion data 110, indicating that the specific image was captured while the patient's jaw was in the position indicated by the specific frame of the motion data 110. In some implementations, the image capture system 107 includes a three-dimensional camera and the image data 109 may include one or more three-dimensional images. Examples of three-dimensional cameras include stereo cameras (e.g., using two or more separate image sensors that are offset from one another). The three-dimensional camera may also include a projector such as a light projector or laser projector that operates to project a pattern on the patient's face. For example, the projector may be offset relative to the camera or cameras so that the images captured by the camera include distortions of the projected pattern caused by the patient's face. Based on these distortions, the three-dimensional structure of portions of the patient's face can be approximated. Various implementations project various patterns such as one or more stripes or fringes (e.g., sinusoidally changing intensity values). In some implementations, the three-dimensional image is captured in relation to the motion capture system 200 or a portion thereof so that the three-dimensional images can be related to the same coordinate system as the motion data.

Still referring to FIG. 1B, the example dental lab 104 includes a 3D scanner 112, the remote computer system 160, a rapid fabrication machine 119, and a denture fabrication station 122. Although shown as a single dental lab, the dental lab 104 may also include multiple dental labs. For example, the 3D scanner 112 can be in a different dental lab than one or more of the other components shown in the dental lab 104. Further, one or more of the components shown in the dental lab 104 may not be physically located in a dental lab. For example, one or more of the 3D scanner 112, remote computer system 160, rapid fabrication machine 119, and denture fabrication station 122 can be physically located in the dental office 102. Additionally, some implementations of the system 100 may not include all of the components shown in the dental lab 104 in FIG. 1B.

The example 3D scanner 112 is a device that can be configured to create a three-dimensional digital representation of the dental impression 108. In some implementations, the 3D scanner 112 generates a point cloud, a polygonal mesh, a parametric model, or voxel data representing the dental impression 108. In some implementations, the 3D scanner 112 generates a digital dental model 114. In some implementations, the 3D scanner 112 includes a laser scanner, a touch probe, and/or an industrial CT scanner. Yet other implementations of the 3D scanner 112 are possible as well. Further, sometimes, the system 100 may not include the 3D scanner 112. For example, where the dental impression station 106 generates a digital dental impression, the 3D scanner 112 may not be included. In these implementations, the dental impression 108 may be the digital dental model 114 or may be used directly to generate the digital dental model 114.

The remote computer system 160 is a system that is configured to generate denture and/or aligner data 118. In some implementations, the data 118 can include three-dimensional digital data that represents a denture and/or aligner component 120 and is in a format suitable for fabrication using the rapid fabrication machine 119. The remote computer system 160 may use the digital dental model 114, the image data 109, and the motion data 110 to generate the data 118. For example, the remote computer system 160 may generate an aligner setup having a geometric form that is shaped to fit a portion of the digital dental model 114 (e.g., a portion fo the model representing a top row of teeth to be moved to a target teeth setup). As another example, the remote computer system 160 may additionally or alternatively generate a denture base having a geometric form that is shaped to fit a portion of the digital dental model 114 (e.g., a portion of the model representing an edentulous region of the patient's dentition). The system 160 may also determine various parameters that are used to generate the data 118 based on the image data 109 and/or the motion data 110. For example, implementations of the system 160 may use various image processing techniques to estimate a vertical dimension parameter from the image data 109. The system 160 can also use the motion data to perform the techniques described in reference to FIGS. 10-13 to generate and assess teeth aligner setups for patients, such as the patient 150.

In some implementations, the remote computer system 160 includes a computing device having one or more user input devices. The system 160 may include computer-aided-design (CAD) software that generates a graphical display of the data 118 and allows an operator to interact with and manipulate the data 118. In some implementations, the system 160 may include a user interface that allows a user to specify or adjust parameters of the denture design, teeth aligner setup, and/or target teeth setup, such as vertical dimension, overbite, overjet, or tip, torque, and rotation parameters for one or more teeth.

For example, the system 160 may include virtual tools that mimic the tools and techniques used by a laboratory technician to physically design teeth aligners or dentures. In some implementations, the system 160 includes a user interface tool to move a digital representation of the patient's dentition (e.g., the digital dental model 114) according to the motion data 110 (which may be similar to a physical articulator). Additionally, the system 160 can include a server that partially or fully automates generation of designs of the data 118, which may use the motion data 110.

The rapid fabrication machine 119 can include one or more three-dimensional printers. Another example of the rapid fabrication machine 119 is stereolithography equipment. Yet another example of the rapid fabrication machine 119 is a milling device, such as a computer numerically controlled (CNC) milling device. In some implementations, the rapid fabrication machine 119 is configured to receive files in STL format. The received files can include teeth aligner design data. Other implementations of the rapid fabrication machine 119 are possible as well.

The rapid fabrication machine 119 is configured to use the data 118 to fabricate a denture and/or aligner component 120. The component 120 can be a physical component that can be made with any suitable material for manufacturing/fabricating aligners. In other implementations, the component 120 can be a mold formed from wax or another material that is to be used indirectly (e.g., through a lost wax casting or ceramic pressing process) to fabricate the denture and/or aligner 124. Further, the component 120 can be formed using laser sintering technology. The rapid fabrication machine 119 may include a 3D printer that fabricates the denture and/or aligner 124 directly from a material that is suitable for placement in the patient's mouth. The rapid fabrication machine 119 may also print parts using multiple materials.

As described herein, the rapid fabrication machine 119 can receive a single file that includes 3D model components (e.g., 3D meshes) that are to be formed using a variety of materials. The rapid fabrication machine 119 may include reservoirs (or spools) of material or filament in multiple colors to allow for printing unitary components. Some implementations of the machine 119 may include three, four, five, or twelve material reservoirs of different colors. As an illustrative example, may include red, yellow, and blue materials in different reservoirs that may be combined to form many different colors, shades of colors, gradients of colors, and/or layering of colors in different zones of teeth and/or gingiva being printed as the denture 124. Some implementations may also include a dark (e.g., black) color material to alter brightness of the combined material. The color may be specified in terms of CYMK (cyan, yellow, magenta, key (black)). Some implementations may also include a white reservoir that can be used to alter the brightness of the material blend. The rapid fabrication machine 119 may include other reservoirs of material that are, for example, colors commonly used in dentures to reduce the need to blend colors.

In some implementations, the rapid fabrication machine 119 can print with a material having a color determined from multiple polygons or vertices. For example, the rapid fabrication machine 119 may determine color on a surface of a polygon using a process similar to Gouraud shading by, for example, variably blending the colors of vertices of the polygon based on position with respect to (distance to) the vertices. The interior of the fabricated parts may be fabricated with a material based on a blend of the surface colors. The interior of the fabricated parts may be fabricated with a material based on other factors, such as cost reduction (e.g., using materials with lower costs), weight reduction (e.g., using materials with lower weights), or other material properties such as strength (e.g., using materials with a desired strength or other material property).

Still referring to FIG. 1B, the denture and/or aligner fabrication station 122 operates to fabricate the denture and/or aligner 124 for the patient. The fabrication station 122 uses the component 120 produced by the rapid fabrication machine 119 in some implementations. In some implementations, the denture and/or aligner 124 is a complete or partial dental appliance to be put inside the patient's mouth. In some implementations, the dental impression 108 is used in the fabrication of the denture and/or aligner 124. In some implementations, the dental impression 108 is used to form a plaster model of the aligner for the patient. Additionally, a model of the dentition of the patient can be generated by the rapid fabrication machine 119. The fabrication station 122 can include equipment and processes to perform some or all of the techniques used in traditional dental laboratories to generate dental appliances. Other implementations of the fabrication station 122 are possible as well.

In some implementations, the denture and/or aligner 124 is seated in the mouth of the patient in the dental therapy station 126 by a dentist. The dentist can confirm that an occlusal surface of the denture and/or aligner 124 is properly defined by instructing the patient to engage in various bites. The dentist can also confirm that the aligner properly fits onto/over the patient's teeth and/or applies an appropriate/desired amount of pressure on particular portions of the patient's teeth.

Additionally, the dental office 102 may be connected to the dental lab 104 via a network. The network may be an electronic communication network that facilitates communication between the dental office 102 and the dental lab 104. An electronic communication network is a set of computing devices and links between the computing devices. The computing devices in the network use the links to enable communication among the computing devices in the network. The network can include routers, switches, mobile access points, bridges, hubs, intrusion detection devices, storage devices, standalone server devices, blade server devices, sensors, desktop computers, firewall devices, laptop computers, handheld computers, mobile telephones, and other types of computing devices.

In various implementations, the network includes various types of links. For example, the network can include one or both of wired and wireless links, including Bluetooth, ultra-wideband (UWB), 802.11, ZigBee, and other types of wireless links. Furthermore, in various implementations, the network is implemented at various scales. For example, the network can be implemented as one or more local area networks (LANs), metropolitan area networks, subnets, wide area networks (such as the Internet), or can be implemented at another scale.

FIG. 2 is a schematic block diagram illustrating an example motion capture system 200 for capturing jaw movement. In this example, the motion capture system 200 includes an imaging system 202, a patient assembly 204, and a motion determining device 206. Also shown in FIGS. 1A-B are a patient and a network.

In some implementations, the imaging system 202 includes an optical sensing assembly 210 and a screen assembly 212. The optical sensing assembly 210 may capture a plurality of images as the patient's jaw moves. For example, the optical sensing assembly 210 may include one or more cameras such as video cameras. In some implementations, the optical sensing assembly 210 captures a plurality of images that do not necessarily include the patient assembly, but can be used to determine the position of the patient assembly 204. For example, the patient assembly 204 may emit lights that project onto surfaces of the screen assembly 212 and the optical sensing assembly 210 may capture images of those surfaces of the screen assembly 212. In some implementations, the optical sensing assembly 210 does not capture images but otherwise determines the position of the projected light or lights on the surfaces of the screen assembly 212.

The screen assembly 212 may include one or more screens. A screen may include any type of surface upon which light may be projected. Some implementations include flat screens that have a planar surface. Some implementations may include rounded screens, having cylindrical (or partially cylindrical) surfaces. The screens may be formed from a translucent material. For example, the locations of the lights projected on the screens of the screen assembly 212 may be visible from a side of the screens opposite the patient assembly 204 (e.g., the screen assembly 212 may be positioned between the optical sensing assembly 210 and the patient assembly 204).

In addition to capturing the images, the imaging system 202 may capture or generate various information about the images. As an example, the imaging system 202 may generate timing information about the images. Although alternatives are possible, the timing information can include a timestamp for each of the images. Alternatively or additionally, a frame rate (e.g., 10 frames/second, 24 frames/second, 60 frames/second) is stored with a group of images. Other types of information that can be generated for the images includes an identifier of a camera, a position of a camera, or settings used when capturing the image.

The patient assembly 204 is an assembly that is configured to be secured to the patient. The patient assembly 204 or parts thereof may be worn by the patient and may move freely with the patient (i.e., at least a part of the patient assembly 204 may, when mounted to the patient, move in concert with patient head movement). In contrast, in at least some implementations, the imaging system 202 is not mounted to the patient and does not move in concert with patient head movement.

In some implementations, the patient assembly 204 may include light emitters (or projectors) that emit a pattern of light that projects on one or more surfaces (e.g., screens of the screen assembly 212), which can be imaged to determine the position of the patient assembly 204. For example, the light emitters may emit beams of substantially collimated light (e.g., laser beams) that project onto the surfaces as points. Based on the locations of these points on the surfaces, a coordinate system can be determined for the patient assembly 204, which can then be used to determine a position and orientation of the patient assembly 204 and the patient's dentition.

In some implementations, the patient assembly 204 includes separate components that are configured to be worn on the upper dentition and the lower dentition and to move independently of each other so that the motion of the lower dentition relative to the upper dentition can be determined. Examples of the patient assembly 204 are illustrated and described throughout, including in FIG. 3.

The motion determining device 206 determines the motion of the patient assembly 204 based on images captured by the imaging system 202. In some implementations, the motion determining device 206 includes a computing device that uses image processing techniques to determine three-dimensional coordinates of the patient assembly 204 (or portions of the patient assembly) as the patient's jaw is in different positions. For example, images captured by the optical sensing assembly 210 of screens of the screen assembly 212 may be processed to determine the positions on the screens at which light from the patient assembly is projected. These positions on the screens of the screen assembly 212 may be converted to three-dimensional coordinates with respect to the screen assembly 212. From those three-dimensional coordinates, one or more positions and orientations of the patient assembly 204 (or components of the patient assembly 204) may be determined.

Based on the determined positions and orientations of the patient assembly 204, some implementations determine the relative positions and movements of the patient's upper and lower dentition. Further, some implementations infer the location of a kinematically derived axis that is usable in modeling the motion of the patient's mandible (including the lower dentition) about the temporomandibular joint. The kinematically derived axis may be a hinge axis or a screw axis. For example, the hinge axis may be derived from a portion of the motion data (e.g., the motion date corresponding to a hinging open/closed of the patient's jaw). The hinge axis location may also be determined based on radiographic imaging such as CBCT data. Additional motion data may be synthesized based on the location of the hinge axis. For example, if the location of the hinge axis is inferred based on motion data corresponding to hinging open/closed, motion data for other bite movements (e.g., excursive or protrusive movements) may be synthesized based on that hinge axis.

FIG. 3 illustrates a block diagram of an example patient assembly 204. In this example, the patient assembly includes a clutch 220 and a reference structure 222. Here, the clutch 220 and the reference structure 222 are not physically connected and can move independently of one another.

The clutch 220 is a device that is configured to couple to a patient's dentition. For example, the clutch 220 may grip any remaining teeth of the dentition of the patient. In some implementations, the clutch 220 may couple to an edentulous region of a patient's dentition or to dental implants that have been placed in edentulous regions of the patient's dentition.

In some implementations, the clutch 220 comprises a dentition coupling device 224 and a position indicator system 228. In some implementations, the clutch 220 is configured to couple to the lower dentition of the patient so as to move with the patient's mandible. In other implementations, the clutch 220 may be configured to couple to the patient's upper dentition so as to move with the patient's maxilla.

The dentition coupling device 224 is configured to removably couple to the patient's dentition. In some implementations, the dentition coupling device 224 rigidly couples to the patient's dentition such that while coupled, the movement of the dentition coupling device 224 relative to the patient's dentition is minimized. Various implementations include various coupling mechanisms.

For example, some implementations couple to the patient's dentition using brackets that are adhered to the patient's teeth with a dental or orthodontic adhesive. As another example, some implementations couple to the patient's dentition using an impression material. For example, some implementations of the dentition coupling device 224 comprise an impression tray and an impression material such as polyvinyl siloxane. To couple the dentition coupling device 224 to the patient's dentition, the impression tray is filled with impression material and then placed over the patient's dentition. As the impression material hardens, the dentition coupling device 224 couples to the patient's dentition.

Alternatively, some implementations comprise a dentition coupling device 224 that is custom designed for a patient based on a three-dimensional model of the patient's dentition. For example, the dentition coupling device 224 may be formed using a rapid fabrication machine. One example of a rapid fabrication machine is a three-dimensional printer, such as the PROJET® line of printers from 3D Systems, Inc. of Rock Hill, South Carolina. Another example of a rapid fabrication machine is a milling device, such as a computer numerically controlled (CNC) milling device. In these implementations, the dentition coupling device 224 may comprise various mechanical retention devices such as clasps that are configured to fit in an undercut region of the patient's dentition or wrap around any remaining teeth.

Implementations of the dentition coupling device 224 may couple to the patient's dentition using a combination of one or more mechanical retention structures, adhesives, and impression materials. For example, the dentition coupling device 224 may include apertures through which a fastening device (also referred to as a fastener) such as a temporary anchorage device may be threaded to secure the dentition coupling device 224 to the patient's dentition, gum tissue, or underlying bone tissue. For example, the temporary anchorage devices may screw into the patient's bone tissue to secure the dentition coupling device 224.

In some implementations, the dentition coupling device 224 includes one or more fiducial markers, such as hemispherical inserts, that can be used to establish a static relationship between the position of the clutch 220 and the patient's dentition. For example, the dentition coupling device 224 may include three fiducial markers disposed along its surface. The location of these fiducial markers can then be determined relative to the patient's dentition such as by capturing a physical impression of the patient with the clutch attached or using imaging techniques such as capturing a digital impression (e.g., with an intraoral scanner) or other types of images of the dentition and fiducial markers. Some implementations of the dentition coupling device 224 do not include fiducial markers. One or more images or a digital impression of the patient's dentition captured while the dentition coupling device 224 is mounted may be aligned to one or more images or a digital impression of the patient's dentition captured while the dentition coupling device 224 is not mounted.

The position indicator system 228 is a system that is configured to be used to determine the position and orientation of the clutch 220. In some implementations, the position indicator system 228 includes multiple fiducial markers. In some examples, the fiducial markers are spheres. Spheres work well as fiducial markers because the location of the center of the sphere can be determined in an image regardless of the angle from which the image containing the sphere was captured. The multiple fiducial markers may be disposed (e.g., non-collinearly) so that by determining the locations of each (or at least three) of the fiducial markers, the position and orientation of the clutch 220 can be determined. For example, these fiducial markers may be used to determine the position of the position indicator system 228 relative to the dentition coupling device 224, through which the position of the position indicator system 228 relative to the patient's dentition can be determined.

Some implementations of the position indicator system 228 do not include separate fiducial markers. In at least some of these implementations, structural aspects of the position indicator system 228 may be used to determine the position and orientation of the position indicator system 228. For example, one or more flat surfaces, edges, or corners of the position indicator system 228 may be imaged to determine the position and orientation of the position indicator system 228. In some implementations, an intraoral scanner is used to capture a three-dimensional model (or image) that includes a corner of the position indicator system 228 and at least part of the patient's dentition while the dentition coupling device 224 is mounted. This three-dimensional model can then be used to determine a relationship between the position indicator system 228 and the patient's dentition. The determined relationship may be a static relationship that defines the position and orientation of the position indicator system 228 relative to a three-dimensional model of the patient's dentition (e.g., based on the corner of the position indicator system 228 that was captured by the intraoral scanner).

In some implementations, the position indicator system 228 includes a light source assembly that emits beams of light. The light source assembly may emit substantially collimated light beams (e.g., laser beams). In some implementations, the light source assembly is rigidly coupled to the dentition coupling device 224 so that as the dentition coupling device 224 moves with the patient's dentition, the beams of light move. The position of the dentition coupling device 224 is then determined by capturing images of where the light beams intersect with various surfaces (e.g., translucent screens disposed around the patient). Implementations that include a light source assembly are illustrated and described throughout.

The reference structure 222 is a structure that is configured to be worn by the patient so as to provide a point of reference to measure the motion of the clutch 220. In implementations where the clutch 220 is configured to couple to the patient's lower dentition, the reference structure 222 is configured to mount elsewhere on the patient's head so that the motion of the clutch 220 (and the patient's mandible) can be measured relative to the rest of the patient's head. For example, the reference structure 222 may be worn on the upper dentition. Beneficially, when the reference structure 222 is mounted securely to the patient's upper dentition, the position of the reference structure 222 will not be impacted by the movement of the mandible (e.g., muscle and skin movement associated with the mandibular motion will not affect the position of the reference structure 222). Alternatively, the reference structure 222 may be configured to be worn elsewhere on the patient's face or head.

In some implementations, the reference structure 222 is similar to the clutch 220 but configured to be worn on the dental arch opposite the clutch (e.g., the upper dentition if the clutch 220 is for the lower dentition). For example, the reference structure 222 shown in FIG. 3 includes a dentition coupling device 230 that may be similar to the dentition coupling device 224, and a position indicator system 234 that may be similar to the position indicator system 228.

In some implementations, the patient assembly 204 includes a gothic arch tracer. For example, the clutch 220 may include one or more tracing components that may move across a surface of the reference structure 222. The tracing components may have adjustable heights.

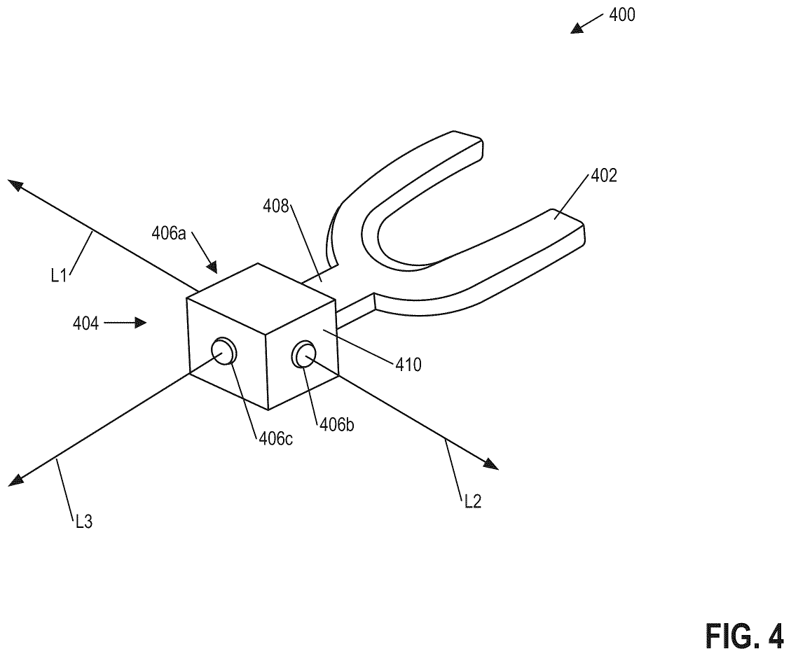

FIG. 4 illustrates an implementation of a clutch 400. The clutch 400 is an example of the clutch 220. In this example, the clutch 400 includes a dentition coupling device 402 and a light source assembly 404, and an extension member 408. The dentition coupling device 402 is an example of the dentition coupling device 224, and the light source assembly 404 is an example of the position indicator system 228.

The light source assembly 404, which may also be referred to as a projector, is a device that emits light beams comprising light that is substantially collimated. Collimated light travels in one direction. A laser beam is an example of collimated light. In some implementations, the light source assembly 404 includes one or more lasers. Although alternatives are possible, the one or more lasers may be semiconductor lasers such as laser diodes or solid-state lasers such as diode-pumped solid-state lasers.

In some implementations, the light source assembly 404 comprises a first, second, and third light emitter. The first and second light emitters may emit substantially collimated light in parallel but opposite directions (i.e., the first and second light emitters may emit light in antiparallel directions) such as to the left and right of the patient when the clutch 400 is coupled to the patient's dentition. In some implementations, the first and second light emitters are collinear or are substantially collinear (e.g., offset by a small amount such as less than 5 micrometers, less than 10 micrometers, less than 25 micrometers, less than 50 micrometers, or less than 100 micrometers). The third light emitter may emit substantially collimated light in a direction of a line that intersects with or substantially intersects with lines corresponding to the direction of the first and second light emitters. Lines that intersect share a common point. Lines that substantially intersect do not necessarily share a common point, but would intersect if offset by a small amount such as less than 5 micrometers, less than 10 micrometers, less than 25 micrometers, less than 50 micrometers, or less than 100 micrometers. In some implementations, the third light emitter emits light in a direction that is perpendicular to the first and second light emitters, such as toward the direction the patient is facing.

In some implementations, the third light emitter emits light in a direction that is offset from the direction of the first light emitter so as to be directed toward the same side of the patient as the direction of the first light emitter. For example, the third light emitter may be offset from the first light emitter by an offset angle that is an acute angle. The third light emitter may be offset from the first light emitter by an offset angle that is less than 90 degrees such that the light emitted by both the first light emitter and the second light emitter intersect with the same screen (e.g., a planar screen having a rectangular shape and being disposed on a side of the patient). The third light emitter may be offset from the first light emitter by an offset angle of between approximately 1 degree to 45 degrees. In some implementations, the offset angle is between 3 degrees and 30 degrees. In some implementations, the offset angle is between 5 degrees and 15 degrees. For example, the offset angle may be less than 10 degrees.

In some implementations, one or more compensation factors are determined to compensate for an offset from the first and second light emitters being collinear, or an offset from the third light emitter emitting light in a direction of a line that intersects with the directions of the first and second light sources. A compensation factor may also be determined for the offset angle of the third light emitter with respect to the first and second light emitters. For example, an offset angle compensation factor may specify the angle between the direction of the third light emitter and a line defined by the first light emitter. In implementations in which the orientation of the third light emitter is directed perpendicular to or substantially perpendicular to the direction of the first light emitter, the offset angle compensation factor may be 90 degrees or approximately 90 degrees. In implementations in which the orientation of the third light emitter is directed toward a side of the patient, the offset angle compensation factor may, for example, be between approximately 5 and 45 degrees. The compensation factors may be determined specifically for each position indicator system manufactured to account for minor variation in manufacturing and assembly. The compensation factors may be stored in a datastore (such as on the motion determining device 206 or on a computer readable medium accessible by the motion determining device 206). Each position indicator system may be associated with a unique identifier that can be used to retrieve the associated compensation factor. The position indicator system 234 may include a label with the unique identifier or a barcode, QR code, etc. that specifies the unique identifier.

Some implementations of the light source assembly 404 include a single light source and use one or more beam splitters such as prisms or reflectors such as mirrors to cause that light source to function as multiple light emitters by splitting the light emitted by that light source into multiple beams. In at least some implementations, the emitted light emanates from a common point. As another example, some implementations of the light source assembly 404 may comprise apertures or tubes through which light from a common source is directed. Some implementations may include separate light sources for each of the light emitters.

In the example of FIG. 3, the light source assembly 404 includes light emitters 406a, 406b, and 406c (referred to collectively as light emitters 406) and a housing 410. The light emitter 406a is emitting a light beam L1, the light emitter 406b is emitting a light beam L2, and the light emitter 406c is emitting a light beam L3. The light beams L1 and L2 are parallel to each other, but directed in opposite directions. The light beam L3 is perpendicular to the light beams L1 and L2. In at least some implementations, the housing 410 is configured to position the light emitters 406 so that the light beams L1, L2, and L3 are approximately coplanar with the occlusal plane of the patient's dentition. Although the light beam L3 is perpendicular to the light beams L1 and L2 in the example of FIG. 3, alternatives are possible.

The housing 410 may be approximately cube shaped and includes apertures through which the light emitters 406 extend. In other implementations, the light emitters do not extend through apertures in the housing 410 and instead light emitted by the light emitters 406 passes through apertures in the housing 410.

In the example of FIG. 4, the dentition coupling device 402 is rigidly coupled to the light source assembly 404 by an extension member 408. The extension member 408 extends from the dentition coupling device 402 and is configured to extend out of the patient's mouth when the dentition coupling device 402 is worn on the patient's dentition. In some implementations, the extension member 408 is configured so as to be permanently attached to the light source assembly 404 such as by being formed integrally with the housing 410 or joined by welding or a permanent adhesive. In other implementations, the extension member 408 is configured to removably attach to the light source assembly 404. Because the light source assembly 404 is rigidly coupled to the dentition coupling device 402, the position and orientation of the dentition coupling device 402 can be determined from the position and orientation of the light source assembly 404.

In some implementations, the housing 410 and the dentition coupling device 402 are integral (e.g., are formed from a single material or are coupled together in a manner that is not configured to be separated by a user). In some implementations, the housing 410 includes a coupling structure configured to removably couple to the extension member 408 of the dentition coupling device 402. In this manner, the dentition coupling device 402 can be a disposable component that may be custom fabricated for each patient, while the light source assembly 404 may be reused with multiple dentition coupling devices. In some implementations, the housing 410 includes a connector that is configured to mate with a connector on the dentition coupling device 402.

Additionally or alternatively, the housing 410 may couple to the dentition coupling device 402 with a magnetic clasp. Some implementations include a registration structure that is configured to cause the housing 410 to join with the dentition coupling device 402 in a repeatable arrangement and orientation. In some implementations, the registration structure comprises a plurality of pins and corresponding receivers. In an example, the registration structure includes a plurality of pins disposed on the housing 410 and corresponding receivers (e.g., holes) in the dentition coupling device 402 (or vice versa). In some implementations, the registration structure comprises a plurality of spherical attachments and a plurality of grooves. In one example, the registration structure includes three or more spherical attachments disposed on the housing 410 and two or more v-shaped grooves disposed on the dentition coupling device 402 that are disposed such that the spherical attachments will only fit into the grooves when the housing 410 is in a specific orientation and position relative to the dentition coupling device 402. In some implementations, the registration structure includes a spring-mounted pin or screw that serves as a detent to impede movement of the housing 410 with respect to the dentition coupling device 402.

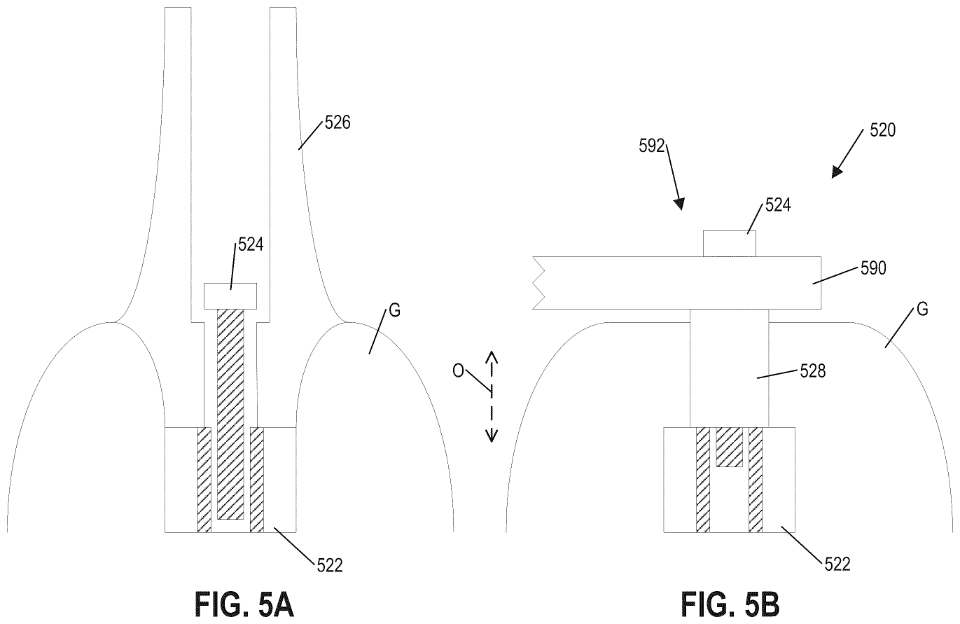

FIGS. 5A-B are cross-sectional side views that illustrate the attachment of an implementation of a dentition coupling device 520 to a dental implant 522. The dentition coupling device 520 is an example of the dentition coupling device 224 or the dentition coupling device 230. The dentition coupling device 520 may include one or more fixed arms and one or more pivotable arms that can be positioned to align with the patient's dentition.

FIG. 5A is a cross-sectional side view that illustrates an implant abutment 526 attached to a dental implant 522 that is disposed in the patient's gingival tissue G. The implant abutment 526 is held in place with an implant screw 524. The implant screw 524 has threads that mate with corresponding threads in a receiver of the dental implant 522. A patient receiving dentures may have several dental implants placed to support and secure the denture.

FIG. 5B is a cross-sectional side view of the dental implant 522 and gingival tissue G with the implant abutment 526 removed and the dentition coupling device 520 attached to the dental implant 522. Here, the implant screw 524 passes through a slot 592 of an arm 590 of the dentition coupling device 520, through an offset 528, and into the dental implant 522. As shown in this figure, at least a portion of the threads of the implant screw 524 are interlocked with the threads of the receiver of the dental implant 522. The offset 528 may be a cylindrical structure that includes an aperture through which a portion of the implant screw 524 may pass. For example, an aperture in the offset 528 may allow passage of the threaded portion of the implant screw 524 but not the head of the implant screw 524. The offset 528 may be sized in the occlusal dimension (O) so as to offset the arm 590 from the gingival tissue G.

Some implementations use a washer to couple the implant screw 524 to the arm 590 (e.g., when an aperture in the arm 590 is larger than the head of the screw). The washer may be formed from a flexible material such as rubber. In some implementations, the arm 590 may be secured to the threads of the receiver of the dental implant 522 with a scanning abutment. A scanning abutment may include a threaded region that is sized to fit into and mate with the threads of the receiver of the dental implant 522. The scanning abutment may also include a fiducial structure that can used to determine a location and orientation of the implant 522 when the scanning abutment is attached. For example, the scanning abutment may be imaged with a component of the image capture system (e.g., an intraoral scanner or a 2D or 3D camera) to determine the locations of the associated dental implant.

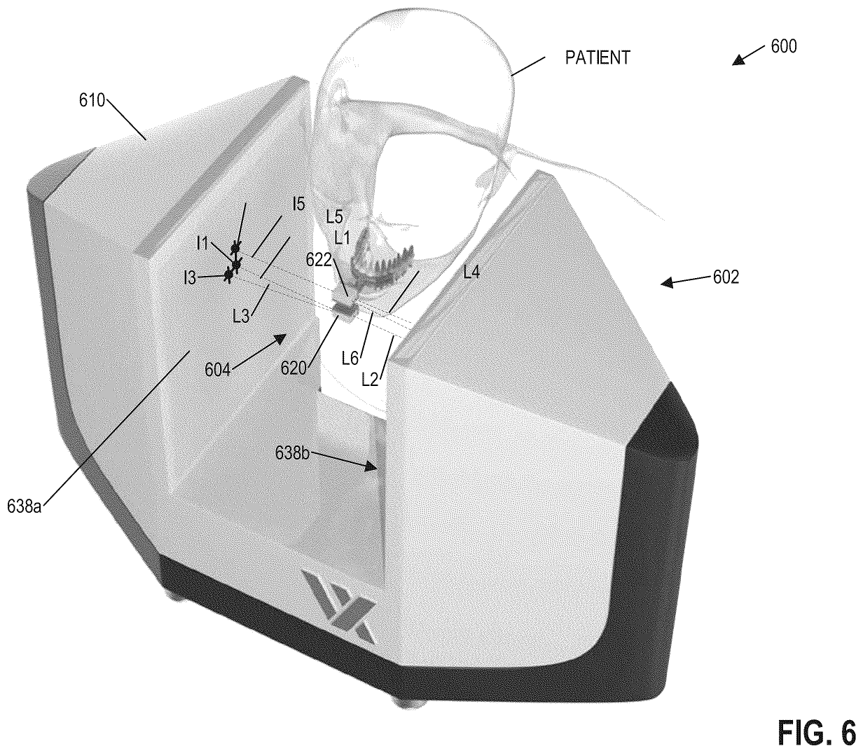

FIG. 6 illustrates an implementation of a motion capture system 600 for capturing jaw movement in which only two screens are used. The motion capture system 600 is an example of the motion capture system 200. The motion capture system 600 includes an imaging system 602 and a patient assembly 604. In this example, the imaging system 602 includes a housing 610. The imaging system also includes screen 638a and a screen 638b (collectively referred to as screens 638), which are positioned so as to be on opposite sides of the patient's face (e.g., screen 638b to the left of the patient's face and screen 638a to the right of the patient's face). In some implementations, a screen framework is disposed within the housing 610 to position the screens 638 with respect to each other and the housing 610.

As can be seen in FIG. 6, this implementation does not include a screen disposed in front of the patient's face. Beneficially, by not having a screen in front of a patient's face, the motion capture system 600 may allow better access to the patient's face by a caregiver. Also shown is patient assembly 604 of the motion capture system 600.

In at least some implementations, the patient assembly 604 includes a clutch 620 and a reference structure 622, each of which include a light source assembly having three light emitters. The clutch 620 is an example of the clutch 220 and the reference structure 622 is an example of the reference structure 222. In FIG. 6, the clutch 620 is attached to the patient's mandible (i.e., lower dentition) and is emitting light beams L1, L2, and L3. Light beams L1 and L3 are directed toward the screen 638a, intersecting at intersection points I1 and I3, respectively. Light beam L2 is directed toward the screen 638b. Although alternatives are possible, in this example, the light beams L1 and L3 are offset from each other by approximately 15 degrees. The light beams L1 and L2 are collinear and directed in opposite directions (i.e., L2 is offset from L1 by 180 degrees).

The reference structure 622 is attached to the patient's maxilla (i.e., upper dentition) and is emitting light beams L4, L5, and L6. Light beams L4 and L6 are directed toward the screen 638b. Light beam L5 is directed toward the screen 638a, intersecting at intersection point 15. Although alternatives are possible, in this example, the light beams L4 and L6 are offset from each other by approximately 15 degrees. The light beams L4 and L5 are collinear and directed in opposite directions (i.e., L4 is offset from L5 by 180 degrees).

As the patient's dentition moves around, the clutch 620 and the reference structure 622 will move in concert with the patient's dentition, causing the light beams to move and the intersection points to change. An optical sensing assembly of the motion capture system 600 (e.g., cameras embedded within the housing 610 of the motion capture system 600 behind the screens 638a and 638b) may capture images of the screens 638 so that the intersection points can be determined.

The location of a first axis associated with the clutch 620 may be identified based on the intersection points from the light beams L1 and L2. An intersection coordinate between the light beams L1 and L3 may then be determined based on the distance between the intersection points I1 and I3 on the screen 638a. For example, the distance from the intersection point I1 along the first axis can be determined based on the distance between the points I1 and I3 and the angle between I1 and I3. As described in more detail elsewhere herein, the angle between I1 and I3 is determined for the clutch 620 and may be stored in a data store, for example, on a non-transitory computer-readable storage medium. Using this distance, the intersection coordinate can be found, which will have a known relationship to the clutch 620 and therefore the patient's dentition. As has been described earlier, a coordinate system for the clutch 620 can be determined based on the intersection points too (e.g., a second axis is defined by the cross product of the first axis and a vector between the intersection points I1 and I3, and a third axis is defined by the cross product of the first axis and the second axis). In a similar manner, the position and orientation of the reference structure 622 can be determined based on the intersection points of the light beams L4, L5, and L6 with the screens 638a and 638b.

In some implementations, three-dimensional coordinate systems for the clutch and the reference structure are determined using only two screens. In some implementations, the motion capture system includes only two screens and the motion capture system does not include a third screen. In some implementations, the imaging system captures images of only two screens. Some implementations identify intersection points using images captured of only two screens. For example, two intersection points from light beams emitted by a reference structure are identified on an image of the same screen.

In some implementations, a light emitter being oriented to emit light in a first direction toward the screen means the light emitter is oriented to emit light in a first direction toward the screen when the light emitter is attached to a patient (or other structure) and positioned for motion tracking with respect to the imaging system.

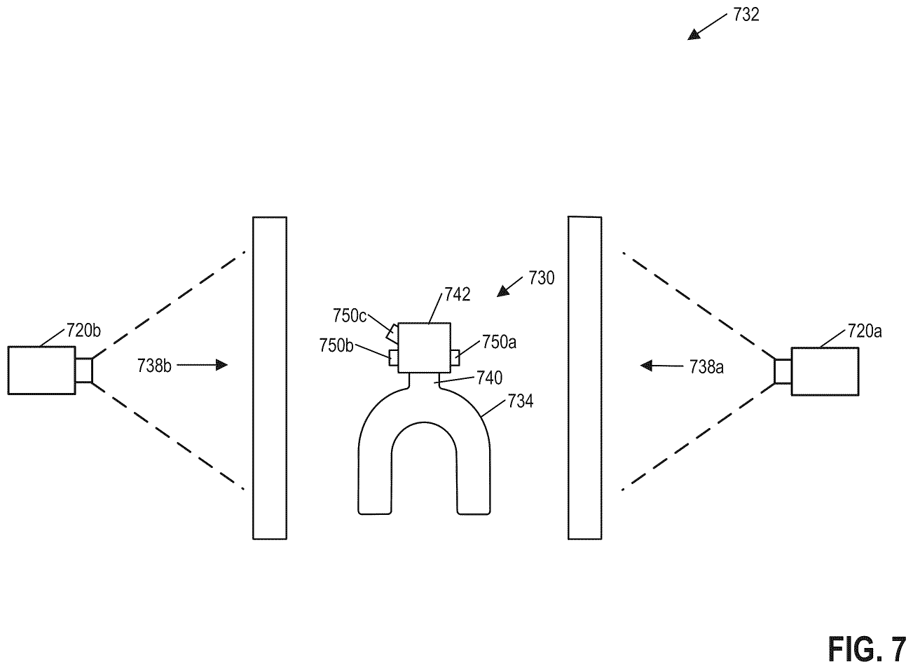

FIG. 7 illustrates a top view of an implementation of a reference structure 730 and an implementation of an imaging system 732. The reference structure 730 is an example of the reference structure 622. The imaging system 732 is an example of the imaging system 602.

The reference structure 730 includes a dentition coupling device 734, an extension member 740, and a light source assembly 742. The dentition coupling device 734 is an example of the dentition coupling device 230 and may be similar to the example dentition coupling devices previously described with respect to implementations of the clutch. The light source assembly 742 is an example of the position indicator system 234. In this example, the light source assembly 742 includes light emitters 750a, 750b, and 750c (collectively referred to as light emitters 750).

The dentition coupling device 734 is configured to removably couple to the dentition of the patient. The dentition coupling device 734 is coupled to the opposite arch of the patient's dentition as the clutch (e.g., the dentition coupling device 734 of the reference structure 730 couples to the maxillary arch when a clutch is coupled to the mandibular arch). In some implementations, the dentition coupling device 734 is coupled to the extension member 740 that is configured to extend out through the patient's mouth when the dentition coupling device 734 is coupled to the patient's dentition. The extension member 740 may be similar to the extension member 408.

The imaging system 732 includes screens 738a and 738b (referred to collectively as screens 738), and cameras 720a and 720b (referred to collectively as cameras 720). In this example, the screen 738a is oriented parallel to the screen 738b. In some implementations, the imaging system 732 may also include a screen framework (not shown) that positions the screens 738 with respect to each other. For example, the screen framework may extend beneath the reference structure 730 and couple to the bottoms of the screens 738. Together, the screens 738 and the screen framework are an example of the screen assembly 212. The cameras 720 are an example of the optical sensing assembly 210.

The screens 738 may be formed from a translucent material so that the points where the light beams emitted by the light source assembly 742 intersect with the screens 738 may be viewed from outside of the screens 738. Images that include these points of intersection may be recorded by the cameras 720. The motion determining device 206 may then analyze these captured images to determine the points of intersection of the light beams with the screens 738 to determine the location of the light source assembly 742. The position of the light source assembly of a clutch (not shown) may be determined in a similar manner.

The cameras 720 are positioned and oriented to capture images of the screens 738. For example, the camera 720a is positioned and oriented to capture images of the screen 738a, and the camera 720b is positioned and oriented to capture images of the screen 738b. In some implementations, the cameras 720 are mounted to the screen framework so that the position and orientation of the cameras 720 are fixed with respect to the screens. For example, each of the cameras 720 may be coupled to the screen framework by a camera mounting assembly. In this manner, the position and orientation of the cameras 720 relative to the screens 738 does not change if the screen framework is moved. In some implementations, the screen framework includes a housing (e.g., as shown at 610 in FIG. 6), within which the cameras 720 are disposed.

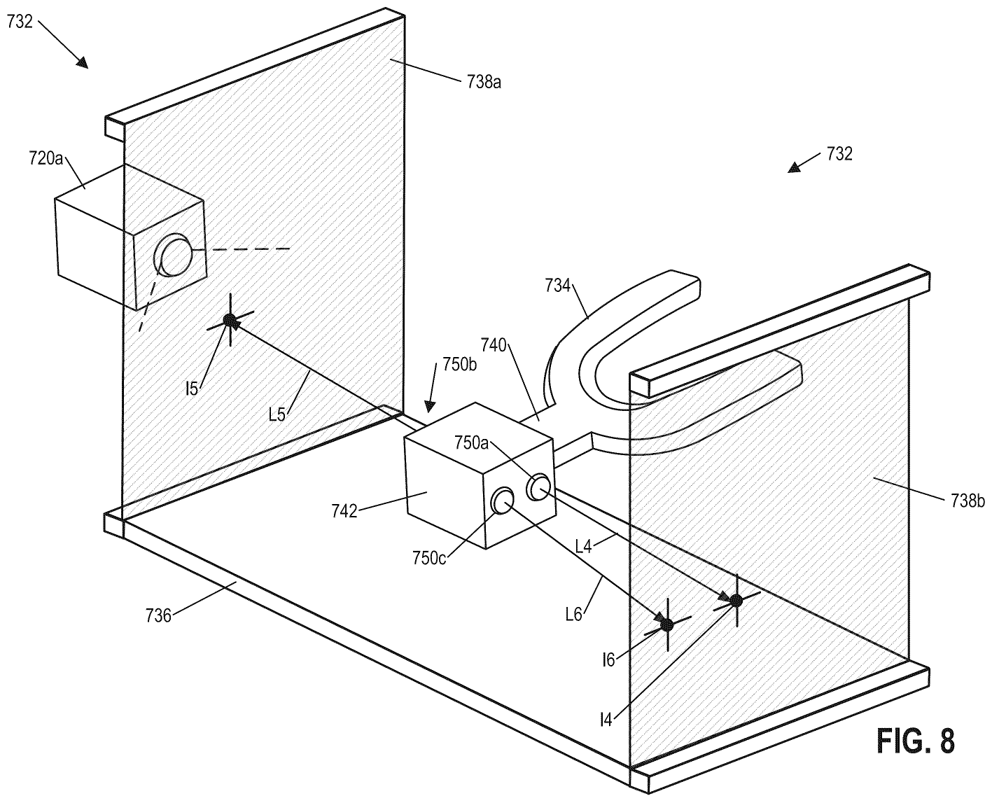

FIG. 8 illustrates a perspective view of the reference structure 730 disposed between the screens 738 of the imaging system 732. The screens 738 are joined together by a screen framework 736 that positions and orients the screens 738 with respect to one another. In this example, the light emitter 750a is emitting a light beam L4, which intersects with the screen 738b at intersection point 14; the light emitter 750b is emitting a light beam L5, which intersects with the screen 738a at intersection point 15; and the light emitter 750c is emitting a light beam L6, which intersects with the screen 738a at intersection point 16. As the position and orientation of the reference structure 730 change relative to the screens 738, the locations of at least some of the intersection points 14, 15, and 16 will change as well.

The camera 720a captures images of the screen 738a, including the intersection point 15 of the light beam L5 emitted by the light emitter 750b. The camera 720a may capture a video stream of these images. Similarly, although not shown in this illustration, the camera 720b captures images of the screens 738b and the intersection points 14 and 16.

The captured images from the cameras 720 are then transmitted to the motion determining device 206. The motion determining device 206 may determine the location of the intersection points 14, 15, and 16, and from those points the location of the light source assembly 742. In some implementations, a point of common intersection for the light beams L4, L5, and L6 is determined based on the location of the intersection points 14, 15, and 16 (e.g., the point at which the light beams intersect or would intersect if extended). Based on the determined locations of the light beams, the location and orientation of the reference structure 730 relative to the screens 738 can be determined.

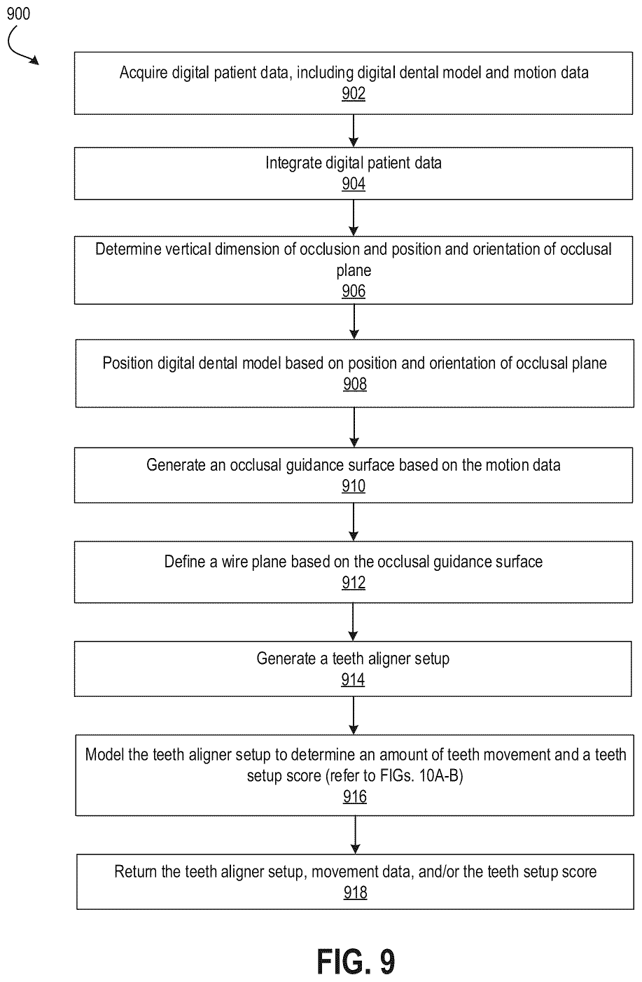

FIG. 9 is a flowchart of an example process 900 for fabricating a teeth aligner setup based on at least motion data. In some implementations, the process 900 is performed by the system 100 described herein, although one or more blocks in the process 900 can also be performed by any other computing system described throughout this disclosure. For illustrative purposes, the process 900 is described from the perspective of a computer system, which may include one or more of the components described in reference to the system 100 in FIGS. 1A-B.

Referring to the process 900 in FIG. 9, at block 902, digital patient data, including motion data and a digital dental model, is acquired for a particular patient. For example, the digital patient data may include imaging data of the patient dentition. The imaging data may be captured using various imaging modalities described herein. In some implementations, the imaging data includes a three-dimensional digital dental model of the patient's dentition. The three-dimensional digital dental model may be captured using an intraoral scanner. The three-dimensional digital dental model may be captured by scanning a physical impression or mold formed from a physical impression using a three-dimensional scanner. Sometimes, the digital dental model can be generated by the computer system and using at least the motion data that is acquired in block 902.

The acquired digital patient data may also include motion data of the patient's jaw. For example, the motion data may be captured using the motion capture system 200. The motion data may represent the patient's jaw moving through various jaw movements including, for example, excursive movements and protrusive movements. The motion data may also represent that patient's jaw position and movement as the patient pronounces specific phonetic sounds such as the “F” sound and the “S” sound. In some implementations, audio or video files may be captured as the patient pronounces the specific sounds. The motion data may map to frames or positions in the video or audio data. Based on sound processing (e.g., audio signal processing) of the audio data or image processing of the video data, various positions in the patient's speech may be identified and the corresponding frame of the motion data may be identified.

The acquired digital patient data may also include one or more anterior facial images of the patient. The anterior facial images may include two-dimensional images or three-dimensional images. In some implementations, the anterior facial images include an image of the patient smiling and an image of the patient with lips in repose (e.g., relaxed). The anterior facial images may also include videos. For example, the videos may include video of the patient performing various jaw movements such as excursive movements and protrusive movements. The videos may also include video of the patient pronouncing specific phonetic sounds such as sibilants (e.g., the “S” sound) or fricatives (e.g., the “F” sound).

The acquired digital patient data may also include other types of patient images captured using imaging modalities such as computed tomography (CT), including cone beam computed tomography (CBCT), ultrasound, and magnetic resonance imaging (MRI).

At block 904, the computer system integrates the digital patient data. For example, the digital patient data may be integrated to a common coordinate system (e.g., positioned relative to the same XYZ axes). Different types of digital patient data may be integrated using different techniques. For example, three-dimensional data sets may be integrated using for example an iterative alignment process such as an iterative closest point technique. In some implementations, multiple types of the digital patient data include fiducial markers. The positions of the fiducial markers may be determined from the digital patient data and used to align one set of digital patient data with another.

In some implementations, the digital patient data includes two-dimensional images captured with a camera of the image capture system 107. A polygon may be generated within the common coordinate system. The two-dimensional images may be mapped to the polygon.

At block 906, a vertical dimension of occlusion and an occlusal plane position and orientation is determined for the patient. The determined vertical dimension of occlusion indicates the desired position of the patient's mandible and maxilla when the patient's jaw is at rest. The vertical dimension of occlusion may correspond to a total distance between edentulous ridges to accommodate dentures with a desired amount of occlusal open space when the patient is at rest. The vertical dimension of occlusion influences the function, comfort, and aesthetics of dentures. The determined occlusal plane may correspond to a plane disposed between the patient's maxilla and mandible that approximately corresponds to where the occlusal surfaces of the patient's teeth meet. The occlusal plane may, for example, be positioned at a desired location of the incisal edge of the patient's upper central incisors, which may be determined from photos of the patient or using a gothic arch tracer. The occlusal plane may be oriented based on the motion data. Although often referred to as an occlusal plane in the denture and dental fields, the occlusal plane need not be precisely planar and may vary from a plane to follow the curve of the patient's lips.

In some implementations, the vertical dimension of occlusion may be specified by a care provider such as dentist or physician. The vertical dimension of occlusion may also be determined based, at least in part, on motion data of the digital patient data. For example, motion data while the patient is pronouncing specific sounds such as sibilants (e.g., the “S” sound) or fricatives (e.g., the “F” sound). A desired vertical dimension of occlusion may be determined from the relative positions of the maxilla and mandible as the sounds are pronounced. The vertical dimension of occlusion may also be determined from a two-dimensional facial image of the digital patient data.

The occlusal plane may, for example, be determined based on applying a ratio to the vertical dimension of occlusion. In some implementations, the occlusal plane may be determined based on the two-dimensional facial image of the digital patient data. For example, the occlusal plane may be positioned so as to align the incisal edges of the upper central incisors with respect to the patient's lips.

In block 908, the digital dental model of the digital patient data is positioned based on the position and orientation of the occlusal plane. For example, a portion of the digital dental model representing the mandibular dental arch may be positioned based on the motion data so as to be positioned at the determined vertical dimension with respect to the maxillary dental arch and so that the denture teeth on the mandibular arch align with the occlusal plane. In some implementations, a frame of the motion data that positions the mandibular dental arch at the determined vertical dimension is identified by the computer system. In some implementations, the mandibular dental arch is rotated about a hinge axis to open to the determined vertical dimension of occlusion. The position of the hinge axis may be inferred based on the motion data.

In some implementations, a user interface is presented by the computer system thar displays the digital dental model, the occlusal plane, or both. The user interface may be configured to receive user input to adjust the vertical dimension of occlusion or the position of the occlusal plane. For example, the user interface may be configured to receive a drag (e.g., click-and-drag or touch-and-drag) input to interactively move the mandibular arch of the digital dental model up or down along an arch defined by the motion data or a hinge axis inferred from the motion data. Similarly, the user interface may be configured to interactively move the occlusal plane along the arch between the mandibular arch and maxillary arch of the digital dental model.

In block 910, the computer system generates an occlusal guidance surface based on the motion data. The occlusal guidance surface may be used to guide the positioning of denture teeth on one of the dental arches, movement of the teeth to achieve a target teeth setup, and/or positioning of a teeth aligner on/over the teeth. The occlusal guidance surface may be generated for one or both of the mandibular arch and the maxillary arch. In some implementations, the occlusal guidance surface is generated for a dental arch by sweeping (or moving) at least a portion of the opposing dental arch according to the motion data. For example, a portion of the opposing dental arch may be swept through one or more of excursive and protrusive movements based on the motion data. In some implementations, the portion of the opposing dental arch may be swept through all of the movements represented in the motion data. In some implementations, a midline polyline segment may be swept according to the motion data. The midline polyline segment may be a cross-section of the opposing dentition at the midline (e.g., middle of the dental arch). The cross-section may be generated by slicing or intersecting a vertically oriented plane through the opposing dentition. In some implementations, the midline polyline segment is not directly based on the opposing dentition. For example, the midline polyline segment may be a line segment on the occlusal plane that extends in the anterior-posterior direction at the midline. As the portion of the opposing dentition is swept according to the motion data, the occlusal guidance surface is generated by the computer system. For example, a midline polyline segment may be duplicated in multiple locations according to the motion data (e.g., the midline polyline segment may be duplicated every 25 micron, every 50 microns, every 100 microns, or another distance). The adjacent midline polyline segments may then be joined to form a surface.

In some implementations, a polygonal surface may be deformed based on the swept midline polyline segment. For example, the polygonal surface may initially be a flat surface that is positioned at the determined occlusal plane location. As the midline polyline segment is swept through different locations, the polygonal surface may be deformed vertically to the midline polyline segment.

Next, the computer system can define a wire plane based on the occlusal guidance surface in block 912. The wire plane can be used to establish placement and design of a teeth aligner setup for the patient.

In block 914, a teeth aligner setup is generated by the computer system. Refer to FIGS. 10A-B for further discussion about generating the teeth aligner setup.

In block 916, the computer system models the teeth aligner setup to determine an amount of teeth movement and/or a teeth setup score. Refer to FIGS. 10A-B for further discussion.

The computer system can return the teeth aligner setup, movement data, and/or the teeth setup score in block 918. Refer to description in FIGS. 1A and 12A-E for further discussion about the returned information and how the returned information may be used, by the computer system and/or by a relevant user, to determine a best teeth aligner setup for the particular patient to achieve the target teeth setup for that patient.

FIGS. 10A-B is a flowchart of a process 1000 for generating and scoring a teeth aligner setup for a patient. The process 1000 can be performed by the remote computer system 160. One or more blocks in the process 1000 can also be performed by other components and/or computing systems described herein. For illustrative purposes, the process 1000 is described from the perspective of a computer system.

Referring to the process 1000 in FIGS. 10A-B, the computer system receives patient oral scan data in block 1002. Refer to blocks A-B (170-172) in FIG. 1A for further discussion.

The computer system can process the data to segment the patient's teeth in block 1004. The computer system can apply machine learning techniques and/or processes to identify each tooth in the oral scan data (e.g., by generating a bounding box or other type of annotation around each tooth). The computer system can also segment upper teeth from bottom teeth and/or identify groups of teeth in the oral scan data. The computer system can segment the teeth per dental arch.

In block 1006, the computer system can generate an automated teeth aligner setup based on the processed data. During setup, the computer system can define a threshold level of consideration for acceptable movement of the patient's teeth by ensuring that roots of the teeth remain in the bone. The computer system can generate an automated teeth aligner setup that is acceptable or a best setup for the particular patient. The acceptable or best setup for the particular patient may not be an acceptable or best setup for other patients. The teeth aligner setup can be generated based on information about the particular patient, such as their preferred or target teeth setup, limitations in which teeth of the patient can be moved and/or how much one or more of the patient's teeth can be moved, and/or practitioner-defined teeth aligner setup information.

For example as part of generating the automated teeth aligner setup, the computer system can identify critical landmarks in the processed data (block 1008). The critical landmarks can include anatomical landmarks including but not limited to cusps, marginal ridges, and/or incisal edges. The critical landmarks can be identified automatically and using one or more rules and/or machine learning techniques. For example, a model can be trained and applied to the processed data to identify the critical landmarks. The model can be trained with image data, oral scan data, and other data that has been annotated (e.g., manually by a relevant user and/or automatically by the computer system) with particular critical landmarks. Sometimes, the identified landmarks can be iteratively fine-tuned.

Additionally or alternatively, the computer system can generate the setup based on the identified landmarks (block 1010).

Additionally or alternatively, the computer system can define a wire plane through the segmented teeth in the setup (block 1012).

Additionally or alternatively, the computer system can apply a coordinate system to each tooth in the setup (block 1014). A Cartesian coordinate system can be applied to each of the segmented teeth in the setup, as shown in FIGS. 12A-E. The coordinate system can be applied automatically by the computer system using the anatomic critical landmarks that were previously identified. Optionally, the coordinate system may be fine-tuned based on user input provided at a computing device of the relevant user. The coordinate system can be used to visualize movement and/or position of each tooth in X, Y, and Z dimensions. The coordinate system can therefore be used to measure, determine, and/or assess changes in movement on an individual tooth level. This can advantageously provide for more accurate determinations of what adjustments can be and/or should be made to the patient's teeth using a teeth aligner.

Additionally or alternatively, the computer system can align each coordinate system with a base of a tooth as defined by the wire plane (block 1016). The computer system can use one or more rules and/or machine learning techniques to automatically align or snap the coordinate system for each tooth to the tooth's base.

In block 1018, the computer system can simulate movement of the patient's teeth from a pre-treatment state to a post-treatment state using the automated teeth aligner setup. For example, the computer system can apply one or more rules and/or machine learning techniques to a digital dental model of the patient's teeth with the automated teeth aligner setup to generate a movement report. The movement report can indicate how much each tooth and/or one or more groups of teeth will or may move once the teeth aligner is applied to the patient's teeth. The movement report can indicate each degree of freedom by which each tooth is expected to move. The computer system can therefore simulate movement in 6 degrees of freedom, for example including but not limited to movement, translation, tip, torque, and rotation. Refer to FIG. 13 for further description about the movement report.

Sometimes, the computer system can generate the movement report or otherwise simulate movement to determine how much each tooth would have to move from the pre-treatment state to the post-treatment state, based on changes that are identified by the computer system to the coordinate system defined for each tooth. The post-treatment state can be a target teeth setup for the particular patient. The post-treatment state can be defined by the relevant practitioner. The post-treatment state can also be defined and/or determined automatically by the computer system and based on various information about the particular patient, as described herein.

The computer system can retrieve one or more models in block 1020. Block 1020 can also be performed at one or more other times in the process 1000.

The computer system can generate a teeth aligner setup score based on applying the model to the movement report resulting from the simulation of block 1018 (block 1022). The model can receive various inputs, as shown in FIG. 13. The model can receive inputs that may include, for example, the automated teeth aligner setup, the digital dental model for the patient, a teeth setup for the pre-treatment state, a teeth setup for the post-treatment state, and optionally other patient information described herein. The model can generate output as a single metric (the setup score) based on processing and correlating the various model inputs. The teeth aligner setup score can be a numeric value, string value, and/or Boolean value. As a numeric value, for example, the score can be assigned in a range of values between and/or including 0 to 100. A higher score value can, in some implementations, indicate a higher difficulty level in achieving the post-treatment state with the teeth aligner setup. A lower score can indicate that the post-treatment state can be more easily and/or more practically achieved with the teeth aligner setup. One or more other criteria can be defined and used for determining the score. Moreover, a standardized scoring criteria can be defined and used with the disclosed techniques so that teeth aligners can be uniformly designed, assessed, and scored by various different entities (including dentists, orthodontists, other care providers, aligner designing companies, aligner manufacturing companies, etc.).

The computer system can return at least the score for the teeth aligner setup in block 1024. For example, the computer system can generate a recommendation of a best setup for the particular patient where the resulting score for that setup is less than a threshold score. The threshold score can be defined for the particular patient and based on patient specific data. In some implementations, the threshold score can be defined for a group or population of patients including the particular patient. As another example, returning the score for the teeth aligner setup can include returning instructions for manufacturing/fabricating aligners based on the setup (e.g., assuming that the score satisfies threshold score criteria to achieve a desired teeth setup for the patient). The returned instructions can be transmitted to a rapid fabrication machine or other system for manufacturing and/or fabricating the aligners according to the teeth aligner setup. Refer to FIGS. 1B-9 for further discussion about manufacturing the aligners.

An iterative feedback loop can be implemented by the computer system after receiving the model output in block 1022. For example, the computer system can optionally set a maximum score that the care provider may be willing to hit with the teeth aligner setup. If the teeth aligner setup surpasses the maximum score, then the computer system can generate one or more recommendations to adjust the teeth aligner setup in order to reduce a resulting score. The computer system can set other limits to determine how much certain teeth can move and restrict various movements of certain teeth so that the teeth aligner setup score can be brought down below a threshold score value or range. The computer system can also automatically iterate through different options and/or adjustments to the teeth aligner setup, then simulate movement to generate another movement report, and assess whether, based on the another movement report, the adjusted teeth aligner setup achieves a lower or more desirable score.

Accordingly, the computer system can modify the teeth aligner setup based on the score satisfying threshold score criteria (block 1026). As part of the threshold score criteria, the computer system may define and/or set limits on how much each tooth or group of teeth may move. As an illustrative example, the limits can vary based on tooth type. In particular, molars may be limited to no more than 0.5 millimeter movement. The computer system can automatically modify the teeth aligner setup in the event that the patient's molars are expected to move more than a threshold amount of desired movement (e.g., 0.5 millimeters). The computer system can automatically modify the teeth aligner setup based on a determination that one or more teeth moved away from a center of an arch by more than or at least a threshold amount (e.g., horizontal movement). As another example, the computer system can automatically modify the teeth aligner setup based on a determination that one or more teeth erupted from the arch by at least a threshold amount and/or at some threshold angle (e.g., vertical movement). The computer system can automatically modify the teeth aligner setup if one or more other criteria are satisfying, in which the criteria may indicate what types of movement are unacceptable, unsafe, or otherwise not preferred for the particular patient.

Sometimes, the computer system can generate a recommended teeth setup for the patient, which can be based on threshold setup criteria (block 1028). The threshold setup criteria can be similar or the same as the threshold score criteria in block 1026. Sometimes, the computer system can generate recommendations for modifications that can be made to the teeth aligner setup, which can then be transmitted to a computing device of the care provider for review, approval, and/or application. Any of the modifications described in block 1026 can, for example, be provided to the care provider as recommendations in block 1028. The care provider can provide user input to select and/or modify one or more of the recommended modifications. The computer system can automatically implement the modification(s) according to the user input.

Sometimes, the computer system can output the teeth aligner setup and/or score at the computing device of a relevant user, such as an orthodontist, dentist, or other care provider (block 1030). Refer to FIGS. 12A-E for further discussion.

The computer system may optionally receive user input indicating at least one modification to the teeth aligner setup based on the score (block 1032). Refer to discussion above in reference to block 1028.

The computer system may optionally modify the teeth aligner setup based on the user input in block 1034. Refer to discussion above in reference to block 1028.

The computer system can then optionally return to block 1018 and continue through blocks 1018-1030 until a desired teeth aligner setup and/or a desired setup score is achieved. In other words, the computer system can simulate movement using the modified teeth aligner setup and assess the movement to determine an updated score for the modified teeth aligner setup. The computer system can determine whether the updated score satisfies threshold score criteria indicating that the modified teeth aligner setup can be used to achieve a desired, preferred, or target teeth setup/outcome for the particular patient. Adjustments, modifications, and/or recommendations can continue to be made to the teeth aligner setup until the corresponding score satisfies the threshold score criteria (which means the desired teeth setup is achievable for the particular patient).

Additionally or alternatively, the computer system can optionally iteratively train the model(s) based on the modified teeth aligner setup, the score, and/or the user input (block 1036). As a result, the model(s) can be continuously improved to more accurately determine preferred teeth aligner setups for patients.

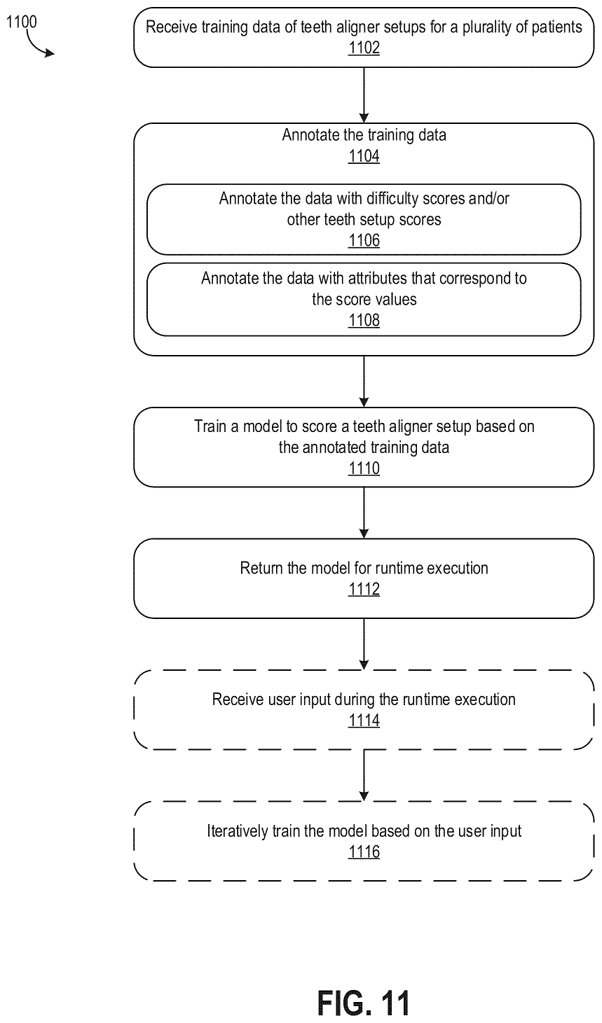

FIG. 11 is a flowchart of a process 1100 for training a model for scoring a teeth aligner setup of a patient. The process 1100 can be performed by the remote computer system 160 described herein. One or more blocks in the process 1100 can also be performed by other computing systems and/or devices. For illustrative purposes, the process 1100 is described from the perspective of a computer system.

Referring to the process 1100 in FIG. 11, the computer system can receive training data of teeth aligner setups for a plurality of patients in block 1102. The training data can include movement data or movement reports, as described throughout this disclosure. The training data can include teeth aligner setups for various patients. The teeth aligner setups can already be scored, such as automatically by the computer system or manually by a human, such as a dentist, orthodontist, or other relevant stakeholder. The training data can include patient cases that have been already scored, for example scored with difficulty index (DI) scores. The DI scores can indicate how difficult it may be for a practitioner to treat the particular patient case. The training data may also include equivalent scores to the DI scores indicating how difficult it may be to treat the patient.

In block 1104, the computer system can annotate the training data. For example, the computer system can annotate the data with difficulty scores and/or other teeth setup scores (block 1106). Sometimes, the data may already be annotated and/or labeled with difficulty scores, such as DI scores. Additionally or alternatively, the computer system can annotate the data with attributes that correspond to the score values (block 1108). For example, the computer system can automatically label particular features in a digital dental model of a patient in the training data that contribute at least a threshold amount to the score value for that patient's case. The computer system can uniquely annotate each feature that contributes to the score value for the patient's case. The annotated features can include particular teeth that would move more than a threshold/desired amount by applying the particular teeth aligner setup to the patient.

The computer system can train a model to score a teeth aligner setup based on the annotated training data (block 1110). As a result, a standardized model can be generated so that it can be applied to a variety of different patient cases having different pre-treatment states and desiring different post-treatment states to accurately score teeth aligner setups to achieve the desired post-treatment states. The model can also be trained to attribute certain features in the training data to higher or lower score values. The model can also be trained to determine how much of the score is attributed to a particular type of movement and/or movement of a particular tooth in the patient's mouth.

The computer system returns the model for runtime execution in block 1112. The model can also be stored in a data store for later retrieval and execution.

Optionally, the computer system can receive user input during the runtime execution in block 1114. The user input can include one or more modifications to the digital dental model for the patient and/or a teeth aligner setup for the patient. The user input can indicate limits for acceptable movement of one or more particular teeth and/or groups of teeth. The user input can also indicate limits on types of movement that may be acceptable. Other user input described herein are also possible.

Optionally, the computer system can iteratively train the model based on the user input (block 1116). The computer system can improve the model by iteratively feeding the user input back into the model at predetermined time intervals and/or whenever the user input is received. As a result, improved model accuracy can result in (i) more accurately identifying features that cause the score value to increase or decrease and (ii) determining more accurate score values based on the received data.

FIG. 12A illustrates an example graphical user interface (GUI) 1200 display for viewing a digital dental model 1202 of a patient's top teeth with an overlaid teeth aligner setup 1206. A dental arch 1204 can be presented as part of the digital dental model 1202 to show a treated outcome for the patient if the patient uses a teeth aligner generated with the teeth aligner setup 1206. A computer system as described herein can apply a coordinate system 1208A-C to each tooth 1210 in the digital dental model 1202. The coordinate system 1208A-C can be used to measure movement of each tooth 1210 in order to achieve the treated outcome shown by the dental arch 1204. For each tooth 1210, the coordinate system 1208A-C includes an X dimension 1208A, a Y dimension 1208B, and a Z dimension 1208C. The coordinate system 1208A-C can be a cartesian coordinate system. As shown in FIG. 12A, the coordinate system 1208A-C can be unique for each tooth 1210.

FIG. 12B illustrates the example GUI 1200 display for viewing a treatment outcome and teeth aligner setup score for the patient's top teeth shown in FIG. 12A. Here, a best fit algorithm, rule, and/or technique has been applied to the digital dental model 1202. In some implementations, an iterative closest points algorithm can be applied to the digital dental model 1202 to best fit each tooth 1210 to the dental arch 1204. As a result, the teeth aligner setup 1206 has been fit to the dental arch 1204 in order to achieve the treated outcome for the patient.

Once the teeth aligner setup 1206 is best fit to the dental arch 1204, the computer system described herein can determine movement of the patient's teeth to achieve the treated outcome. For example, the computer system simulates movement and generates a movement report, as described herein and in particular in reference to FIG. 13. The movement report can include numeric values or other values indicating how much each of the coordinates in the coordinate system 1208A-C for each tooth 1210 changed in various degrees of freedom (e.g., 6 degrees of freedom). Thus, the movement report can indicate how each tooth rotated, torqued, tilted, tipped, translated, etc. to move from the pre-treatment state to the post-treatment state (e.g., the treated outcome or desired teeth setup for the particular patient. The computer system can provide the movement report to a model as input in order to determine and generate a single metric, such as the teeth aligner setup score described throughout this disclosure. Accordingly, a teeth aligner setup assessment 1216 can be presented in the GUI 1200 to the relevant user, such as a dentist.

The teeth aligner setup assessment 1216 can include a variety of information, including but not limited to a score, recommendation, setup difficulty level, and/or score explanation. The score can be a type of difficulty score (e.g., a standardized DI score), which can indicate a difficulty level associated with treating the patient according to the teeth aligner setup 1206. As described herein, the higher numeric value for the score, the more difficulty to treat the patient. The score can be used by the dentist or other relevant user to determine whether to perform the alignment for the patient with the teeth aligner setup 1206. The recommendation can include one or more recommendations about whether the teeth aligner setup 1206 should be used for the patient. The recommendation can include one or more suggested modifications to adjust the teeth aligner setup 1206 so that the score can be lowered and the adjusted teeth aligner setup 1206 can be more feasible for the particular patient. The score explanation can include annotations and/or other information about why the score was determined by the computer system. The score explanation can include annotations as to the rationale behind the score value. As an illustrative example, the score value can be higher or above some threshold value because one or more molars were identified as moving more than a threshold amount with the teeth aligner setup 1206. As another illustrative example, the score can be lower or less than some threshold value because a desired teeth alignment can be achieved with the teeth aligner setup 1206 without moving one or more teeth beyond a threshold amount.

One or more of the information presented in the assessment 1216 can also be converted, by the computer system, into dental terminology that describes the teeth aligner setup 1206, the treated outcome, and/or one or more recommendations/modifications that can be accepted, implemented, or otherwise reviewed by the relevant user, such as a dentist.

The GUI 1200 can also present selectable options, such as buttons, for receiving user feedback/input. For example, an option 1212 can be selected by the user to view the movement report or other movement data that was measured and used to determine the score for the teeth aligner setup 1206. As another example, an option 1214 can be selected by the user to make one or more adjustments to the teeth aligner setup 1206.

FIG. 12C illustrates an example GUI 1220 display for viewing a digital dental model 1222 of a patient's bottom teeth with an overlaid teeth aligner setup 1226. A dental arch 1224 can be presented as part of the digital dental model 1222 to show a treated outcome for the patient if the patient uses a teeth aligner generated with the teeth aligner setup 1226. A computer system as described herein can apply a coordinate system 1228A-C to each tooth 1230 in the digital dental model 1222. The coordinate system 1228A-C can be used to measure movement of each tooth 1230 in order to achieve the treated outcome shown by the dental arch 1224. For each tooth 1230, the coordinate system 1228A-C includes an X dimension 1228A, a Y dimension 1228B, and a Z dimension 1228C. The coordinate system 1228A-C can be a cartesian coordinate system. As shown in FIG. 12B, the coordinate system 1228A-C can be unique for each tooth 1230.

FIG. 12D illustrates the example GUI 1220 display for viewing a treatment outcome and teeth aligner setup score for the patient's bottom teeth shown in FIG. 12C. Here, a best fit algorithm, rule, and/or technique has been applied to the digital dental model 1222, as described in reference to FIG. 12B. Once the teeth aligner setup 1226 is best fit to the dental arch 1224 for the patient's bottom teeth, the computer system described herein can determine movement of the patient's teeth to achieve the treated outcome. For example, the computer system simulates movement and generates a movement report, as described herein and in particular in reference to FIG. 13. The computer system can provide the movement report to a model as input in order to determine and generate a single metric, such as the teeth aligner setup score described throughout this disclosure. Accordingly, a teeth aligner setup assessment 1240 can be presented in the GUI 1220 to the relevant user, such as a dentist.

The teeth aligner setup assessment 1240 can include a variety of information, including but not limited to a score, a recommendation, a setup difficulty, and/or a score explanation. The GUI 2200 can also include selectable options, such as an option 1242 to view the movement data and/or a selectable option 1244 to adjust the teeth aligner setup. Refer to FIG. 12B for further description about the different information that can be presented in the GUI 1220.

Moreover, as shown in FIG. 12D, one or more of the teeth in the digital dental model 1222 can be annotated in some indicia based on how that tooth contributed to the score value. In the example of FIG. 12D, a second left molar 1230 is highlighted in an indicia 1232 in the digital dental model 1222 because the second left molar 1230 was identified, by the computer system, as moving more than a threshold amount that is acceptable for molars (in association with the particular patient or across a population of patients). As a result, the computer system can identify that the procedure may be too difficult for the particular patient because the second left molar 1230 would have too much movement, which can have detrimental health and/or safety effects on the patient.

The indicia 1232 can be a highlighting, glow effect, change in color, pattern, hatching, or other graphical element/indication that can be applied by the computer system to overlay a portion of the digital dental model 1222 in the GUI 1200 that includes the particular tooth 1230. The indicia 1232 can advantageously direct the relevant user's attention to quickly and accurately identify what aspect of the teeth aligner setup 1226 can or should be adjusted to improve the score and therefore make the alignment procedure more feasible for the patient. After all, the user can determine one or more modifications to the teeth aligner setup 1226 that may reduce the amount of movement that was determined or projected for the molar 1230. Sometimes, the computer system can also generate one or more suggestion s for adjusting the teeth aligner setup 1226 that may result in reducing the amount of movement of the molar 1230. Once any adjustments or modifications are made to the teeth aligner setup 1226, the computer system can re-simulate movement of the teeth in the digital dental model 1222 and generate an updated score for the teeth aligner setup 1226. The updated score can be presented in the GUI 1220. The user can iterate through the process described above until a desired outcome is achieved (e.g., the desired outcome being a desired teeth aligner setup 1226, a desired amount of movement of one or more particular teeth, a desired post-treatment teeth setup).

Although the indicia 1232 is described in reference to the patient's bottom teeth in FIG. 12D, the indicia 1232 can also be applied by the computer system to one the patient's upper teeth shown and described in the FIGS. 12A-B. Moreover, although the teeth aligner setup assessment 1240 is presented in the same GUI 1220 as the digital dental model 1222, the assessment 1240 information can be presented in one or more other GUIs, separate GUIs, and/or pop-out windows or notifications. Similarly, the assessment 1216 information can be presented in different GUIs than the digital dental model 1202 in FIG. 12B.

FIG. 12E illustrates an example GUI 1250 display for receiving user input to adjust a teeth aligner setup to achieve a desired treatment outcome. The digital dental model 1202 can be presented in the same GUI 1250 as one or more data input fields for adjusting the teeth aligner setup. Sometimes, the data input fields can be presented in a different GUI and/or in a pop-out window. The user can provide input into any of the data input fields in order to make modifications and/or set limits for modifying the teeth aligner setup. As the user inputs the modifications and/or defines the limits, the computer system described herein can automatically adjust the teeth aligner setup and/or automatically adjust a post-treatment teeth setup for the particular patient. Sometimes, the computer system can implement the adjustments at another time, such as after the user completes providing all of their input into the data input fields. The GUI 1250 can then be updated to reflect the modifications that were made to the teeth aligner setup based on the user input. Sometimes, the modifications and/or defined limits may not be implemented by the computer system until the user selects an option (e.g., a button) to apply those modifications. Sometimes, once the modifications are applied, the computer system can re-simulate movement of the patient's teeth according to the modified teeth aligner setup and determine an updated score for the teeth aligner setup. The computer system can present the simulated movement information and/or the updated score in the GUI 1250 or another GUI presented at the user's computing device.

In the illustrative example of the GUI 1250, a teeth aligner setup assessment 1252 can be presented. The assessment 1252 can include at least the determined score value for the teeth aligner setup of the patient. The assessment 1252 may also include any other information presented and described in reference to FIGS. 12B and 12D. The GUI 1250 can also present data input fields 1254, 1256, and 1258. One or more additional or fewer data input fields can also be presented in the GUI 1250 to provide the user with additional modification capabilities.

The data input field 1254 can be used by the user to input a target score for the teeth aligner setup. The user-inputted target score value can be used by the computer system to determine one or more adjustments that can be made to the teeth aligner setup in order to achieve the user-inputted target score value. The computer system can generate one or more recommendation adjustments and present those recommendations to the user. The user can then select one or more of the recommendations for the computer system to automatically implement. Sometimes, the computer system can automatically implement one or more of the recommended adjustments without first receiving user input selecting the adjustment(s) to be made.

The data input field 1256 can be used by the user to input one or more types of movements to limit. The user can also provide input indicating how much the user would like each type of movement to be limited. The user input can indicate any one or more of the 6 degrees of freedom/rotation by which the teeth in the digital dental model 1202 can be moved in order to achieve the desired tooth setup. The computer system can generate one or more adjustments that can be made to the teeth aligner setup to improve the score value for the teeth aligner setup while abiding by the limits that are defined by the user in the data input field 1256.

The data input field 1258 can be used by the user to input one or more types of teeth, groups of teeth, and/or particular teeth of which to limit adjustments/movements. The computer system can generate adjustments that can be made to the teeth aligner setup to improve the score value for the setup while abiding by the limits that are defined by the user in the data input field 1258.

The GUI 1250 can also present one or more selectable options 1260, 1262, 1264, 1266, and 1268. One or more additional or fewer selectable options with different features/functionality can also be presented in the GUI 1250. Selecting any of the options 1260, 1262, 1264, 1266, and 1268 can cause another GUI to be presented at the user's computing device and/or a pop-out window to be displayed in the GUI 1250.

The option 1260 can be selected by the user to view an adjusted teeth aligner setup. The user can therefore select the option 1260 in order to view adjustments to the setup that were automatically made by the computer system based on the user-provided input in the data input fields 1254, 1256, and/or 1258.

The option 1262 can be selected by the user to score the adjusted teeth aligner setup. For example, the computer system can automatically adjust the teeth aligner setup and/or the user can manually adjust the setup using one or more controls and other GUIs presented at the user's computing device. Once the setup is adjusted, selecting the option 1262 can cause the computer system to re-simulate movement of the teeth in the digital dental model 1202 according to the adjusted teeth aligner setup and score the adjusted teeth aligner setup based on the resulting movement data. The updated score can be presented in a same GUI as the score that was determined for the previous teeth aligner setup. As another example, the updated score can be presented in a different GUI. Sometimes, the updated score can be presented in the same GUI as the previous score, along with the previous teeth aligner setup and the adjusted teeth aligner setup.

The option 1264 can be selected by the user to view the movement data. The user can view the movement data for the current teeth aligner setup. The user can view the movement data for the re-simulation of movement for the adjusted teeth aligner setup. The movement data can be presented in another GUI and/or in a pop-out window in the GUI 1250.

The option 1266 can be selected by the user to adjust the teeth aligner setup. For example, the user can interact with one or more teeth in the digital dental model 1202 to make adjustments to the teeth aligner setup. The user can click and drag on one or more teeth to rotate or otherwise move the teeth in one or more degrees of freedom/rotation. The user can also select one or more selectable options to make adjustments to the teeth in the digital dental model 1202. For example, the user can select one or more computer-recommended adjustments to be implemented.

The option 1268 can be selected by the user to view one or more adjustment recommendations for the teeth aligner setup. Selecting the option 1268 can cause the adjustment recommendations to be presented in a pop-out window in the GUI 1250 and/or another GUI. Selecting the option 1268 can cause the computer system to present similar information as described in reference to the option 1260.

FIG. 13 is a conceptual diagram illustrating runtime execution of a teeth aligner setup scoring model 1302 using the disclosed techniques. The model 1302 can be trained to receive movement data 1300 as input (block A, 1306). Sometimes, the model 1302 can also receive other training inputs as described throughout this disclosure. The model 1302 can process the movement data 1300 in order to generate output 1304 (block B, 1308). The output 1304 can include, for example, a score for a particular teeth aligner setup (e.g., a standardized difficulty index score indicating a difficulty of performing an alignment process with the teeth aligner setup), projected, simulated, or expected movement results of a patient's teeth based on the teeth aligner setup (which can be determined from the movement data 1300 that is generated as a result of simulating movement of the patient's teeth in a digital dental model with the teeth aligner setup; this may include indications of one or more features and/or teeth movements most significant in affecting the determined score value), and/or one or more recommendations for adjusting the teeth aligner setup and/or performing an alignment procedure for the patient using the teeth aligner setup. Refer to description throughout this disclosure for additional details and information about the output 1304.

The movement data 1300 can include different types of recorded movement as a result of simulating movement of the patient's teeth in the patient's digital dental model using the teeth aligner setup. The movement data 1300 can indicate movement of each tooth relative to one or more other teeth and/or groups of teeth in one or more degrees of freedom/rotation. The movement data 1300 can additionally or alternatively indicate movement of a group of teeth, a dental arch, or any other combination of teeth. In some implementations, the movement data 1300 can indicate movement of each tooth relative to a coordinate system assigned to the particular tooth (refer to the coordinate system described in reference to FIGS. 12A-D).

The movement data 1300 can be in the form of a tooth movement report (e.g., .MHT and/or .CVS formats) that quantifies movement for each tooth based on the tooth's coordinate system before and after applying the teeth aligner setup (e.g., before and after best fitting the patient's teeth to a desired outcome/teeth setup). Reported values in the movement data 1300 can include one or more of 6 degrees of freedom. The reported values may also be described or otherwise transcribed into dental terminology, including but not limited to buccal-lingual, mesial-distal, occlusal-gingival, tip, torque, rotate, etc. Angular differences, which can be expressed in degrees (as a non-limiting example), may be represented in the movement data 1300. As shown in the movement data 1300, it can be assumed that rotation of teeth occurs first, followed by tip, and lastly by torque. Linear differences, which can be expressed in millimeters (as a non-limiting example), can indicate, in the movement data 1300, translation of each tooth coordinate system origin. Accordingly, linear translations and angular changes for each tooth are determined relative to each tooth's coordinate system.

The illustrative movement data 1300 in FIG. 13 can include, but is not limited to, mesial-distal translation data 1300A, buccal-lingual translation data 1300B, occlusal-gingival translation data 1300C, rotation change data 1300D, tip change data 1300E, and torque change data 1300N. One or more additional or alternative types of movements can be simulated and reported in the movement data 1300. Moreover, in some implementations, the movement data 1300 can be determined for one or more groups of teeth in addition to or instead of determining movement on an individual tooth level.

The mesial-distal translation data 1300A can show movement of each tooth. Mesial translation can indicate movement of a tooth towards a front of the patient's mouth. Distal translation can indicate movement of a tooth towards a back of the patient's mouth. Positive distances shown in the data 1300A can indicate mesial translations while negative distances shown in the data 1300A can indicate distal translations.

The buccal-lingual translation data 1300B can show movement of each tooth. Buccal translation can indicate movement of a tooth towards the patient's cheek while lingual translation can indicate movement of a tooth towards an inside of the patient's mouth. Positive distances shown in the data 1300B can indicate buccal translations. Negative distances in the data 1300B can indicate lingual translations.

The occlusal-gingival translation data 1300C can show other movements of each tooth. Occlusal translation can indicate movement of a tooth away from bite surfaces in the patient's mouth. Gingival translation can indicate movement of a tooth towards the patient's gums. Positive distances shown in the data 1300C can indicate occlusal translations while negative distances can indicate gingival translations.

The rotation change data 1300D can show determined/simulated rotations of each tooth. For example, the data 1300D can indicate rotations of a tooth around a z-axis of the tooth. For example, the buccal surface can toward towards a midline in the patient's mouth, and this rotation may be reflected in the data 1300D. Positive angles in the data 1300D can indicate mesial rotations while negative angles can indicate distal rotations.

The tip change data 1300E can show how each tooth tips as a result of the simulated movement. The data 1300E can indicate how, for example, a crown may tip towards the midline, which can be between central incisors. The data 1300E can also indicate, as an example, how the crown may tip away from the midline and towards a back of the patient's mouth. Positive angles reflected in the data 1300E can indicate mesial tips of teeth while negative angles can indicate distal tips.

The torque change data 1300N can show how each tooth torques as a result of the simulated movement. The data 1300N can indicate how, for example, a tooth tips positively towards a cheek and/or tips negatively towards an inside of the patient's mouth. Positive angles reflects in the data 1300N can describe a buccal torque while negative angles can describe a lingual torque.

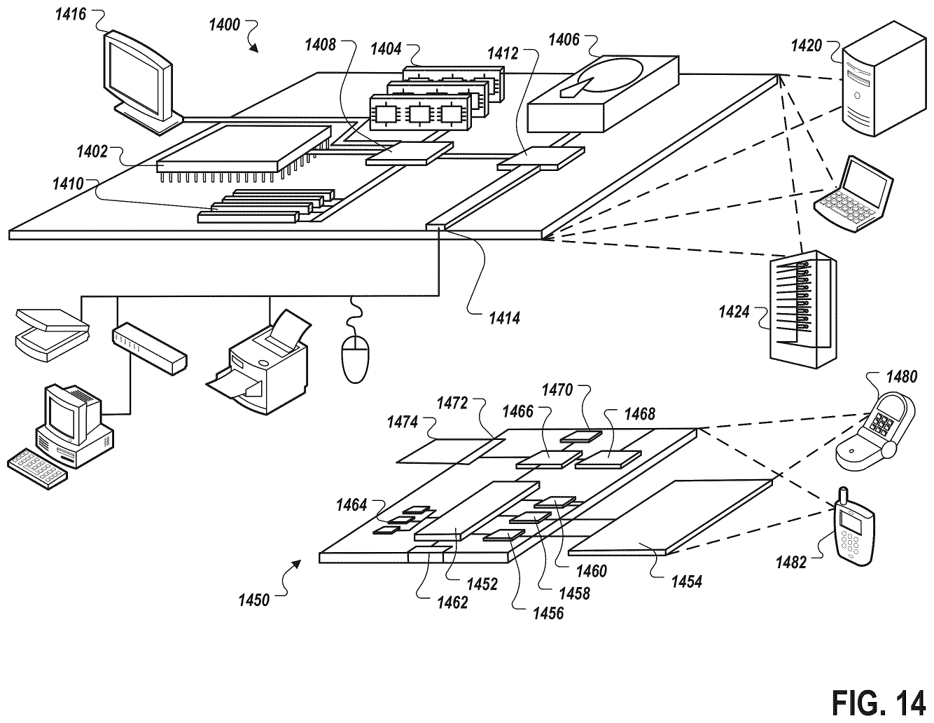

FIG. 14 shows an example of a computing device 1400 and an example of a mobile computing device that can be used to implement the techniques described here. The computing device 1400 is intended to represent various forms of digital computers, such as laptops, desktops, workstations, personal digital assistants, servers, blade servers, mainframes, and other appropriate computers. The mobile computing device is intended to represent various forms of mobile devices, such as personal digital assistants, cellular telephones, smart-phones, and other similar computing devices. The components shown here, their connections and relationships, and their functions, are meant to be exemplary only, and are not meant to limit implementations of the inventions described and/or claimed in this document.

The computing device 1400 includes a processor 1402, a memory 1404, a storage device 1406, a high-speed interface 1408 connecting to the memory 1404 and multiple high-speed expansion ports 1410, and a low-speed interface 1412 connecting to a low-speed expansion port 1414 and the storage device 1406. Each of the processor 1402, the memory 1404, the storage device 1406, the high-speed interface 1408, the high-speed expansion ports 1410, and the low-speed interface 1412, are interconnected using various busses, and can be mounted on a common motherboard or in other manners as appropriate. The processor 1402 can process instructions for execution within the computing device 1400, including instructions stored in the memory 1404 or on the storage device 1406 to display graphical information for a GUI on an external input/output device, such as a display 1416 coupled to the high-speed interface 1408. In other implementations, multiple processors and/or multiple buses can be used, as appropriate, along with multiple memories and types of memory. Also, multiple computing devices can be connected, with each device providing portions of the necessary operations (e.g., as a server bank, a group of blade servers, or a multi-processor system).

The memory 1404 stores information within the computing device 1400. In some implementations, the memory 1404 is a volatile memory unit or units. In some implementations, the memory 1404 is a non-volatile memory unit or units. The memory 1404 can also be another form of computer-readable medium, such as a magnetic or optical disk.

The storage device 1406 is capable of providing mass storage for the computing device 1400. In some implementations, the storage device 1406 can be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product can also contain instructions that, when executed, perform one or more methods, such as those described above. The computer program product can also be tangibly embodied in a computer- or machine-readable medium, such as the memory 1404, the storage device 1406, or memory on the processor 1402.

The high-speed interface 1408 manages bandwidth-intensive operations for the computing device 1400, while the low-speed interface 1412 manages lower bandwidth-intensive operations. Such allocation of functions is exemplary only. In some implementations, the high-speed interface 1408 is coupled to the memory 1404, the display 1416 (e.g., through a graphics processor or accelerator), and to the high-speed expansion ports 1410, which can accept various expansion cards (not shown). In the implementation, the low-speed interface 1412 is coupled to the storage device 1406 and the low-speed expansion port 1414. The low-speed expansion port 1414, which can include various communication ports (e.g., USB, Bluetooth, Ethernet, wireless Ethernet) can be coupled to one or more input/output devices, such as a keyboard, a pointing device, a scanner, or a networking device such as a switch or router, e.g., through a network adapter.

The computing device 1400 can be implemented in a number of different forms, as shown in the figure. For example, it can be implemented as a standard server 1420, or multiple times in a group of such servers. In addition, it can be implemented in a personal computer such as a laptop computer 1422. It can also be implemented as part of a rack server system 1424. Alternatively, components from the computing device 1400 can be combined with other components in a mobile device (not shown), such as a mobile computing device 1450. Each of such devices can contain one or more of the computing device 1400 and the mobile computing device 1450, and an entire system can be made up of multiple computing devices communicating with each other.

The mobile computing device 1450 includes a processor 1452, a memory 1464, an input/output device such as a display 1454, a communication interface 1466, and a transceiver 1468, among other components. The mobile computing device 1450 can also be provided with a storage device, such as a micro-drive or other device, to provide additional storage. Each of the processor 1452, the memory 1464, the display 1454, the communication interface 1466, and the transceiver 1468, are interconnected using various buses, and several of the components can be mounted on a common motherboard or in other manners as appropriate.

The processor 1452 can execute instructions within the mobile computing device 1450, including instructions stored in the memory 1464. The processor 1452 can be implemented as a chipset of chips that include separate and multiple analog and digital processors. The processor 1452 can provide, for example, for coordination of the other components of the mobile computing device 1450, such as control of user interfaces, applications run by the mobile computing device 1450, and wireless communication by the mobile computing device 1450.

The processor 1452 can communicate with a user through a control interface 1458 and a display interface 1456 coupled to the display 1454. The display 1454 can be, for example, a TFT (Thin-Film-Transistor Liquid Crystal Display) display or an OLED (Organic Light Emitting Diode) display, or other appropriate display technology. The display interface 1456 can comprise appropriate circuitry for driving the display 1454 to present graphical and other information to a user. The control interface 1458 can receive commands from a user and convert them for submission to the processor 1452. In addition, an external interface 1462 can provide communication with the processor 1452, so as to enable near area communication of the mobile computing device 1450 with other devices. The external interface 1462 can provide, for example, for wired communication in some implementations, or for wireless communication in other implementations, and multiple interfaces can also be used.

The memory 1464 stores information within the mobile computing device 1450. The memory 1464 can be implemented as one or more of a computer-readable medium or media, a volatile memory unit or units, or a non-volatile memory unit or units. An expansion memory 1474 can also be provided and connected to the mobile computing device 1450 through an expansion interface 1472, which can include, for example, a SIMM (Single In Line Memory Module) card interface. The expansion memory 1474 can provide extra storage space for the mobile computing device 1450, or can also store applications or other information for the mobile computing device 1450. Specifically, the expansion memory 1474 can include instructions to carry out or supplement the processes described above, and can include secure information also. Thus, for example, the expansion memory 1474 can be provide as a security module for the mobile computing device 1450, and can be programmed with instructions that permit secure use of the mobile computing device 1450. In addition, secure applications can be provided via the SIMM cards, along with additional information, such as placing identifying information on the SIMM card in a non-hackable manner.

The memory can include, for example, flash memory and/or NVRAM memory (non-volatile random access memory), as discussed below. In some implementations, a computer program product is tangibly embodied in an information carrier. The computer program product contains instructions that, when executed, perform one or more methods, such as those described above. The computer program product can be a computer- or machine-readable medium, such as the memory 1464, the expansion memory 1474, or memory on the processor 1452. In some implementations, the computer program product can be received in a propagated signal, for example, over the transceiver 1468 or the external interface 1462.

The mobile computing device 1450 can communicate wirelessly through the communication interface 1466, which can include digital signal processing circuitry where necessary. The communication interface 1466 can provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication can occur, for example, through the transceiver 1468 using a radio-frequency. In addition, short-range communication can occur, such as using a Bluetooth, WiFi, or other such transceiver (not shown). In addition, a GPS (Global Positioning System) receiver module 1470 can provide additional navigation- and location-related wireless data to the mobile computing device 1450, which can be used as appropriate by applications running on the mobile computing device 1450.

The mobile computing device 1450 can also communicate audibly using an audio codec 1460, which can receive spoken information from a user and convert it to usable digital information. The audio codec 1460 can likewise generate audible sound for a user, such as through a speaker, e.g., in a handset of the mobile computing device 1450. Such sound can include sound from voice telephone calls, can include recorded sound (e.g., voice messages, music files, etc.) and can also include sound generated by applications operating on the mobile computing device 1450.

The mobile computing device 1450 can be implemented in a number of different forms, as shown in the figure. For example, it can be implemented as a cellular telephone 1480. It can also be implemented as part of a smart-phone 1482, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

While this specification contains many specific implementation details, these should not be construed as limitations on the scope of the disclosed technology or of what may be claimed, but rather as descriptions of features that may be specific to particular implementations of particular disclosed technologies. Certain features that are described in this specification in the context of separate implementations can also be implemented in combination in a single implementation in part or in whole. Conversely, various features that are described in the context of a single implementation can also be implemented in multiple implementations separately or in any suitable subcombination. Moreover, although features may be described herein as acting in certain combinations and/or initially claimed as such, one or more features from a claimed combination can in some cases be excised from the combination, and the claimed combination may be directed to a subcombination or variation of a subcombination. Similarly, while operations may be described in a particular order, this should not be understood as requiring that such operations be performed in the particular order or in sequential order, or that all operations be performed, to achieve desirable results. Particular implementations of the subject matter have been described. Other implementations are within the scope of the following claims.

1. A method for determining a teeth setup score for a teeth aligner setup of a patient, the method comprising:
receiving, by a computer system, oral scan data for a patient, wherein the oral scan data includes at least one of (i) a dental impression generated by a dental impression station, (ii) image data of the patient's mouth generated by an image capture system, and (iii) motion data of the patient's jaw movement generated by a motion capture system;
generating, by the computer system, a teeth aligner setup for the patient based on the oral scan data;
simulating, by the computer system, movement of one or more teeth of the patient using the teeth aligner setup;
generating, by the computer system, a movement report based on the simulated movement;
determining, by the computer system, a teeth setup score for the teeth aligner setup based on providing the movement report as input to a machine learning model; and
returning, by the computer system, the teeth setup score for presentation in a graphical user interface (GUI) display of a computing device of a relevant user.

2. The method of claim 1, wherein returning, by the computer system, the teeth setup score further comprises generating instructions that, when executed by the computing device, cause the computing device to:
output, in the GUI display, the teeth setup score and the teeth aligner setup, wherein the teeth aligner setup is displayed as visually overlaying a digital dental model of the patient's mouth;
receive user input indicating at least one modification to the teeth aligner setup; and
transmit the user input to the computer system.

3. The method of claim 2, further comprising:
receiving, by the computer system and from the computing device, the user input; adjusting, by the computer system, the teeth aligner setup based on the user input;
re-simulating, by the computer system, movement of the patient's teeth using the adjusted teeth aligner setup; and
determining, by the computer system, an updated teeth setup score for the adjusted teeth aligner setup.

4. The method of claim 2, wherein outputting the teeth setup score comprises outputting information about at least one tooth according to the movement report that had a greatest impact on the determined teeth setup score.

5. The method of claim 1, further comprising generating, by the computer system, a digital dental model for the patient based on the oral scan data.

6. The method of claim 1, wherein the teeth setup score indicates a difficulty level that corresponds to performing an alignment procedure on the patient with the teeth aligner setup.

7. The method of claim 1, wherein a higher teeth setup score indicates a greater difficulty level with performing an alignment procedure on the patient with the teeth aligner setup and a lower teeth setup score indicates a lower difficulty level with performing the alignment procedure on the patient with the teeth aligner setup.

8. The method of claim 1, further comprising generating, by the computer system, instructions that, when executed by a rapid fabrication machine, causes the rapid fabrication machine to manufacture a teeth aligner dental appliance for the patient based on the teeth aligner setup.

9. (canceled)

10. The method of claim 1, wherein simulating, by the computer system, movement of one or more teeth of the patient using the teeth aligner setup comprises simulating movement of the patient's teeth from a pre-treatment state to a post-treatment state, wherein the pre-treatment state is a current state of the patient's teeth according to the oral scan data and the post-treatment state is achieved using the teeth aligner setup.

11. The method of claim 1, wherein the movement report includes at least one of: mesial-distal translation data, buccal-lingual translation data, occlusal-gingival translation data, rotation change data, tip change data, and torque change data.

12. The method of claim 1, wherein the movement report includes data about at least one degree of freedom of movement for each tooth in the oral scan data for the patient.

13. The method of claim 12, further comprising applying, by the computer system, a coordinate system to each tooth in the oral scan data for the patient, wherein the at least one degree of freedom of movement for each tooth is determined relative the coordinate system applied to the tooth.

14. The method of claim 1, further comprising:
determining, by the computer system, whether the teeth setup score exceeds a threshold score value;
generating, by the computer system and based on a determination that the teeth setup score exceeds the threshold score value, at least one recommendation for adjusting the teeth aligner setup, wherein adjusting the teeth aligner setup causes at least one change in teeth movement that results in a lower teeth setup score than the teeth setup score that exceeds the threshold score value; and
returning, by the computer system, the at least one recommendation for presentation in the GUI display of the computing device.

15. The method of claim 14, further comprising automatically adjusting, by the computer system and responsive to determining that the teeth setup score exceeds a second threshold score value, the teeth aligner setup based on the at least one recommendation, wherein the second threshold score value is higher than the threshold score value.

16. The method of claim 1, wherein generating, by the computer system, a teeth aligner setup for the patient based on the oral scan data comprises:
segmenting each tooth in the oral scan data;
identifying critical landmarks for each segmented tooth;
generating the teeth aligner setup based on the critical landmarks;
defining a wire plane through the segmented teeth in the teeth aligner setup;
applying a coordinate system to each segmented tooth in the teeth aligner setup; and
aligning each coordinate system with a base of each segmented tooth, wherein the base of the segmented tooth is defined by the wire plane.

17. The method of claim 1, wherein the machine learning model was trained, by the computer system, using a process comprising:
receiving training data of teeth aligner setups for a plurality of patients;
annotating the training data with at least one of (i) difficulty scores having relationships with the teeth aligner setups and (ii) attributes that correspond to the difficulty scores;
training the model to score a teeth aligner setup based on the annotated training data; and
returning the model for runtime execution.

18. A system for scoring a teeth aligner setup for a patient, the system comprising:
a computing device having processors and memory, wherein the computing device is configured to:
present, in a graphical user interface (GUI) display, a digital dental model of a patient, wherein the digital dental model represents a post-treatment teeth setup for the patient;
present, in the GUI display, a teeth aligner setup as a graphical visual overlaying at least a portion of the digital dental model, wherein the teeth aligner setup represents a pre-treatment teeth setup for the patient;
present, in the GUI display and for each tooth in the teeth aligner setup, a coordinate system;
transmit, to a computing system, a request for a teeth setup score based on the teeth aligner setup overlaying the digital dental model;
receive, from the computing system, data indicating at least the teeth setup score, wherein the teeth setup score is determined, by the computing system, based on (i) simulating movement of teeth in the teeth aligner setup to teeth in the digital dental model representing the post-treatment teeth setup for the patient to generate a teeth movement report and (ii) providing the teeth movement report as input to a machine learning model that was trained to generate the teeth setup score; and
present, in the GUI display, the teeth aligner setup as visually overlaying a different portion of the digital dental model, wherein the different portion of the digital dental model is determined based on the teeth movement report; and
present, in the GUI display the teeth setup score.

19. (canceled)

20. The system of claim 18, wherein the computing device is further configured to:
receive second user input indicating at least one adjustment to the teeth aligner setup based on the teeth setup score;
transmit the second user input to the computing system;
receive, from the computing system, data indicating (i) an adjusted teeth aligner setup based on the user input and (ii) an updated teeth setup score based on re-simulating movement of the teeth in the adjusted teeth aligner setup relative the teeth in the digital dental model; and
present, in the GUI display, the adjusted teeth aligner setup as visually overlaying the digital dental model and the updated teeth setup score.

21. The system of claim 18, wherein the teeth setup score is presented in at least one of (i) another GUI display and (ii) a pop-out window overlaying at least a portion of the GUI display.

22. The system of claim 18, wherein the computing device is further configured to present, in the GUI display, at least one of: (i) a recommendation of whether the teeth aligner setup should be used for the patient, the recommendation being based on whether the teeth setup score satisfies threshold alignment criteria, (ii) a difficulty level associated with performing an alignment procedure with the teeth aligner setup, the difficulty level being a string value that corresponds to a numeric value of the teeth setup score, and (iii) a score explanation indicating at least one movement associated with at least one tooth of the teeth aligner setup that satisfied threshold movement criteria.
